(19) United States
(12) Patent Application Publication
(10) Pub. No.: US 2004/0058955 A1
(43) Pub. Date: Mar. 25, 2004

(54) Polymorphs of fexofenadine hydrochloride

(76) Inventors: Ben-Zon Dolitzky, Petach Tiqva (IL); Shlomit Wizel, Petah Tiqva (IL); Barnaba Krochmal, Jerusalem (IL); Dov Diller, Jerusalem (IL); Irwin Gross, Jerusalem (IL)

Correspondence Address: KENYON & KENYON, ONE BROADWAY, NEW YORK, NY, 10004, US

(21) Appl. No.: 10/661,259

(22) Filed: Sep. 12, 2003

Related U.S. Application Data
(62) Division of application No. 10/118,807, filed on Apr. 8, 2002.
(60) Provisional application No. 60/282,521, filed on Apr. 9, 2001.
(60) Provisional application No. 60/307,752, filed on Jul. 25, 2001.
(60) Provisional application No. 60/314,396, filed on Aug. 23, 2001.
(60) Provisional application No. 60/336,930, filed on Nov. 8, 2001.
(60) Provisional application No. 60/339,041, filed on Dec. 7, 2001.
(60) Provisional application No. 60/344,114, filed on Dec. 28, 2001.
(60) Provisional application No. 60/361,780, filed on Mar. 4, 2002.
(60) Provisional application No. 60/363,482, filed on Mar. 11, 2002.

(51) Int. Cl. C07D 211/22; A61K 31/445

(52) U.S. Cl. 514/317; 546/241

(57) ABSTRACT

The present invention provides novel crystal forms of fexofenadine hydrochloride Forms V, VI and VIII through XV and processes for their preparation and preparation of amorphous form and other crystalline forms of fexofenadine hydrochloride. Forms XIV and XV are solvates of ethyl acetate, while Form IX is anhydrous, but can be crystallized as a solvate of MTBE or cyclohexane. The forms are useful for administration to humans and animals to alleviate symptoms caused by histamine. The present invention further provides pharmaceutical compositions of the new crystalline forms.

### CROSS-REFERENCE TO RELATED APPLICATIONS

This application claims the benefit of provisional applications Serial Nos. 60/282,521, filed Apr. 9, 2001; 60/307,752, filed Jul. 25, 2001; 60/314,396, filed Aug. 23, 2001; 60/336,930, filed Nov. 8, 2001; 60/339,041, filed Dec. 7, 2001; 60/344,114, filed Dec. 28, 2001; 60/361,780, filed Mar. 4, 2002 and 60/363,482, filed Mar. 11, 2002, all of which are incorporated herein by reference.

### FIELD OF THE INVENTION

The present invention relates to the solid state chemistry of fexofenadine hydrochloride and its use as an active pharmaceutical agent.

### BACKGROUND OF THE INVENTION

4-[4-[4-(hydroxydiphenylmethyl)-1-piperidinyl]-1-hydroxybutyl]-α,α-dimethylbenzeneacetic acid of formula (I) (fexofenadine) is an H₁ receptor antagonist and a useful antihistaminic drug. It has low permeability into central nervous system tissues and weak antimuscarinic activity, causing it to have few systemic side effects.

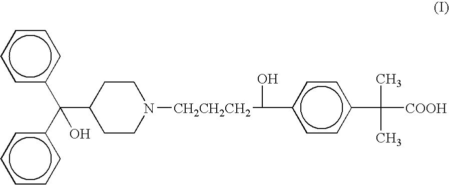

The antihistamic activity of fexofenadine was first disclosed in U.S. Pat. No. 4,254,129, incorporated herein by reference. According to the '129 patent, fexofenadine can be prepared starting from ethyl α,α-dimethylphenyl acetate and 4-chlorobutyroyl chloride, which are reacted under Freidel-Crafts conditions. Chloride is displaced from the Freidel-Crafts product with α,α-diphenyl-4-piperidinemethanol to give 4-[4-[4-(hydroxydiphenylmethyl)-1-piperidinyl]-1 -oxobutyl]-α,α-dimethylbenzeneacetate, which is isolated as its hydrochloride salt. The ketone is then reduced with PtO/H₂ and the ester group is hydrolyzed to yield fexofenadine hydrochloride.

Other methods of preparing fexofenadine are discussed in U.S. Pat. Nos. 5,578,610, 5,589,487, 5,581,011, 5,663,412, 5,750,703, 5,994,549, 5,618,940, 5,631375, 5,644,061, 5,650,516, 5,652,370, 5,654,433, 5,663,353, 5,675,009, 5,375,693 and 6,147,216.

The present invention relates to the solid state physical properties of fexofenadine hydrochloride, resulting from the conformation and orientation of molecules in the unit cell.

U.S. Pat. Nos. 5,738,872, 5,932,247 and 5,855,912, incorporated herein by reference, describe four crystal forms of fexofenadine hydrochloride which were designated Forms I-IV. According to the '872 and related patents, Forms II and IV are hydrates and Forms I and III are anhydrous. Each form was characterized by its melting point, onset of endotherm in the DSC profile, and PXRD. Form I is reported to have a capillary melting point range of 196-201° C., a DSC endotherm with onset between 195-199° C. and a powder X-ray diffraction (“PXRD”) pattern with d-spacings of 14.89, 11.85, 7.30, 6.28, 5.91, 5.55, 5.05, 4.96, 4.85, 4.57, 4.45, 3.94, 3.89, 3.84, 3.78, 3.72, 3.63, 3.07, 3.04, 2.45 Å. Form II is reported to have a capillary melting point range of 100-105° C., a DSC endotherm with onset between 124-126° C. and a PXRD pattern with d-spacings of 7.8, 6.4, 5.2, 4.9, 4.7, 4.4, 4.2, 4.1, 3.7, 3.6, 3.5 Å. Form III is reported to have a capillary melting point range of 166-171° C., a DSC endotherm with onset at 166° C. and a PXRD pattern with d-spacings of 8.95, 4.99, 4.88, 4.75, 4.57, 4.47, 4.46, 3.67, 3.65 Å. In Example 2, Form IV is reported to undergo decomposition at 115-116° C. In the general written description, a DSC endotherm with onset at 146° C. is reported. Form IV is reported as having a PXRD pattern with d-spacings of 10.38, 6.97, 6.41, 5.55, 5.32, 5.23, 5.11, 4.98, 4.64, 4.32, 4.28, 4.12, 4.02, 3.83, 3.65, 3.51, 3.46 and 2.83 Å.

The '872 patent discusses methods of interconverting Forms I-IV. Aqueous recrystallization of Form I can be used to produce Form II. Water-minimizing recrystallization or azeotropic distillation of either Form II or Form IV can yield Form I. Form III is reported to be accessible by water minimizing recrystallization of Form II. Crystal digestion of Form III can be used to obtain Form I. Forms II and IV can be obtained directly by sodium borohydride reduction of 4-[4-[4-(hydroxydiphenylmethyl)-1-piperidinyl]-1-oxobutyl]-α,α-dimethylbenzeneacetate as described in Examples 1 and 2.

In Example 2 of the '129 patent, fexofenadine base is isolated after platinum oxide reduction of the ketone precursor by evaporating the 4:1 ethanol:methanol reaction solvent and recrystallizing the residue from butanone or a methanol-butanone mixture. The product is reported to melt in the range of 185-187° C.

International Publication No. WO 00/71124 A1, discloses that amorphous fexofenadine hydrochloride can be prepared by lyophilizing or spray drying a solution of fexofenadine hydrochloride. The product is characterized by its IR spectrum and a featureless PXRD pattern.

The present invention provides new crystal forms of fexofenadine hydrochloride and processes for preparation of various forms of fexofenadine hydrochloride.

### SUMMARY OF THE INVENTION

In one aspect the present invention provides a process for preparing amorphous fexofenadine hydrochloride comprising the steps of: preparing a solution of fexofenadine hydrochloride in tetrahydrofuran (“THF”); removing a portion of THF from the solution; adding a C₅ to a C₁₂ saturated hydrocarbon to THF to form an upper and a lower layer, wherein the lower layer is oily; separating the upper layer from the lower layer; and drying the lower layer to obtain amorphous fexofenadine. Amorphous fexofenadine hydrochloride can also be prepared by a process comprising preparing a solution of fexofenadine hydrochloride in an organic solvent and removing the solvent.

In another aspect the present invention provides fexofenadine hydrochloride Form V, having a PXRD pattern with peaks at about 15.9, 16.8, 17.2, 20.9, 21.5, 21.8±0.2 degrees two theta, which can be prepared by a process comprising the steps of: preparing a solution of fexofenadine hydrochloride in a mixture of water and an alcohol selected from the group consisting of methanol, isopropanol, ethanol and 1-butanol; forming a precipitate; and separating the precipitate.

In another aspect the present invention provides fexofenadine hydrochloride Form VI, having a PXRD pattern with peaks at about 15.7, 16.1, 17.0, 17.3, 18.6, 18.8±0.2 degrees two theta, which can be prepared by a process comprising the steps of preparing a solution of fexofenadine hydrochloride in a mixture of water and 1-propanol; forming a precipitate; and separating the precipitate. Fexofenadine hydrochloride Form VI also can be prepared by a process comprising the steps of: preparing a solution of fexofenadine hydrochloride in THF (“THF”); adding water to the solution to form a precipitate; and separating the precipitate.

In another aspect the present invention provides a process for preparing fexofenadine hydrochloride Form II comprising the step of heating fexofenadine hydrochloride Form V or Form VI from about 40° C. to about 80° C.

In another aspect the present invention provides fexofenadine hydrochloride Form VIII having a PXRD pattern with peaks at about 8.5, 11.0, 11.4, 13.4, 13.8, 17.1, 20.0, 21.5±0.2 degrees two theta and a DSC thermogram with endothermic peaks at about 84° C. and about 142° C., which can be prepared by a process comprising the steps of: preparing a solution of fexofenadine base (syn. free base) in a basic aqueous solvent; adding hydrochloric acid to the solution to form a precipitate; and separating the precipitate.

In another aspect the present invention provides fexofenadine hydrochloride Form IX, having a PXRD pattern with peaks of about 4.7, 9.3, 17.4, 18.2, 19.4, 19.6, 21.6 and 24.0±0.2 degrees two theta and a DSC thermogram with an endotherm at about 139° C., which can be prepared by a process comprising the steps of: preparing a solution of fexofenadine hydrochloride in acetone; adding the solution to an antisolvent to form a precipitate; and separating the precipitate.

In another aspect the present invention provides fexofenadine hydrochloride Form IX-MTBE solvate, characterized by a DTG profile with endotherms at about 100° C. and about 125° C., which can be prepared by a process comprising the steps of adding fexofenadine hydrochloride Form IX to MTBE to form the solvate; and separating the solvate.

In another aspect the present invention provides fexofenadine hydrochloride Form IX-cyclohexane solvate, characterized by a DTG profile with endotherms at about 99° C. to about 110° C. and about 140° C. to about 150° C., which can be prepared by a process comprising the steps of adding fexofenadine hydrochloride Form IX to cyclohexane to form the solvate; and separating the solvate.

In another aspect the present invention provides fexofenadine hydrochloride Form X. Fexofenadine hydrochloride Form X has a PXRD pattern with peaks at about 4.2, 8.0, 9.3, 14.2, 16.0, 16.8, 17.6, 18.8, 20.0, 20.6, 21.7, 22.9, 23.8, 24.2 and 25.4±0.2 degrees two theta and a DTG profile with a maximum endotherm at about 100° C. and a minor endotherm at about 138° C., which can be prepared by a process comprising the steps of: preparing a solution of fexofenadine hydrochloride in methanol, and optionally adding dichloromethane to said solution; adding a C₅ to a C₁₂ saturated hydrocarbon to the solution to form a precipitate; and separating the precipitate. Another process for preparing fexofenadine hydrochloride Form X comprises the steps of: preparing a solution of fexofenadine hydrochloride in methanol; removing the methanol to obtain a residue; adding a mixture of methanol and an anti-solvent to the residue to form a precipitate; and separating the precipitate.

In another aspect the present invention provides fexofenadine hydrochloride Form XI, having a PXRD pattern with peaks at about 8.7, 14.5, 14.9, 16.6, 17.2, 18.3, 19.5, 21.2, 22.1 and 23.3±0.2 degrees two theta, which can be prepared by a process comprising the steps of: preparing a solution of fexofenadine hydrochloride in methanol; adding the solution to toluene to form a precipitate; and separating the precipitate.

In another aspect the present invention provides fexofenadine hydrochloride Form XII, having a PXRD peaks at about 5.2, 7.9, 8.1, 12.1, 18.5, 19.0±0.2 degrees two theta and a FTIR spectrum with peaks at about 731, 845, 963, 986, 999, 1072, 1301, 1412 and 3313 cm⁻¹, which can be prepared by a process comprising the steps of: preparing a solution of fexofenadine hydrochloride in ethanol; removing the ethanol to obtain a residue; adding a mixture of ethanol and toluene to the residue to form a precipitate; and separating the precipitate.

In another aspect the present invention provides fexofenadine hydrochloride Form XIII, having a PXRD pattern with peaks at about 5.5, 6.8, 16.0, 16.3±0.2 degrees two theta, a DSC thermogram with an endothermic peak at about 185-195° C., a FTIR spectrum with peaks at about 1249, 1365, 1719 and 3366cm⁻¹, which can be prepared by a process comprising heating fexofenadine hydrochloride Form XII for a sufficient amount of time to obtain substantially fexofenadine hydrochloride Form XII.

In another aspect the present invention provides for fexofenadine hydrochloride ethyl acetate solvates, designated Form XIV and Form XV.

Form XIV is characterized by a PXRD diffraction pattern with peaks at about 5.4, 5.7, 10.9, 11.4, 11.6±0.2 degrees two theta, a DSC thermogram with an endothermic peak at about 100° C., a FTIR spectrum with peaks at about 634.3, 699.5, 1335, 1359 and 1725 cm⁻¹, wherein the peaks at 1335, 1359 and 1725 are split and can be prepared by a process comprising the steps of: dissolving fexofenadine hydrochloride in methanol; removing methanol to obtain a residue; adding a mixture of methanol and toluene to the residue to form a precipitate;separating the precipitate; adding the precipitate to ethyl acetate to form the solvate and separating the solvate; Fexofenadine hydrochloride Form XIV can be prepared by another process comprising triturating fexofenadine hydrochloride From X in ethyl acetate.

Form XV produces a PXRD pattern with peaks at about 5.5, 5.8, 16.4, 16.9, 18.4±0.2 degrees two theta. Its DSC thermogram has an endothermic peak at about 140° C. Form XV can be prepared by dissolving fexofenadine hydrochloride in ethanol; removing ethanol to obtain a residue; adding a mixture of toluene and ethanol to the residue to form a precipitate; separating the precipitate; adding the precipitate to ethyl acetate to form the solvate; and separating the solvate. Form XV can be prepared by triturating fexofenadine hydrochloride Form XII in ethyl acetate.

In another aspect, the present invention provides pharmaceutical compositions of the new polymorphs and their methods of administration.

### BRIEF DESCRIPTION OF THE FIGURES

FIG. 1 is a PXRD pattern of fexofenadine hydrochloride Form V.

FIG. 2 is a PXRD pattern of fexofenadine hydrochloride Form VI.

FIG. 3 is a PXRD pattern of fexofenadine hydrochloride Form VIII.

FIG. 4 is a differential scanning calorimetric (DSC) thermogram of fexofenadine hydrochloride Form VIII.

FIG. 5 is a DTG profile (thermogravimetric analysis) of Form VIII plotting weight loss versus temperature.

FIG. 6 is a PXRD pattern of fexofenadine hydrochloride Form IX.

FIG. 7 is a DSC thromogram of fexofenadine hydrochloride Form IX.

FIG. 8 is a DTG profile of fexofenadine hydrochloride Form X plotting weight loss versus temperature.

FIG. 9 is a PXRD pattern of fexofenadine hydrochloride Form X.

FIG. 10 is a PXRD pattern of fexofenadine hydrochloride Form XI.

FIG. 11 is a PXRD pattern for fexofenadine hydrochloride Form XII.

FIG. 12 is a FTIR spectrum for fexofenadine hydrochloride Form XII.

FIG. 13 is a PXRD pattern for fexofenadine hydrochloride Form XIII.

FIG. 14 is a DSC thermogram for fexofenadine hydrochloride Form XIII.

FIG. 15 is a FTIR spectrum of fexofenadine hydrochloride Form XIII.

FIG. 16 is a PXRD pattern for fexofenadine hydrochloride Form XIV.

FIG. 17 is a DSC thermogram for fexofenadine hydrochloride Form XIV.

FIG. 18 is a PXRD pattern for fexofenadine hydrochloride Form XV.

FIG. 19 is a DSC thermogram for fexofenadine hydrochloride Form XV.

FIG. 20 is a FTIR spectrum for fexofenadine hydrochloride Form XIV.

FIG. 21 is a FTIR spectrum for fexofenadine hydrochloride Form XV.

FIG. 22 is a PXRD pattern for fexofenadine hydrochloride amorphous prepared by the process of Example 3.

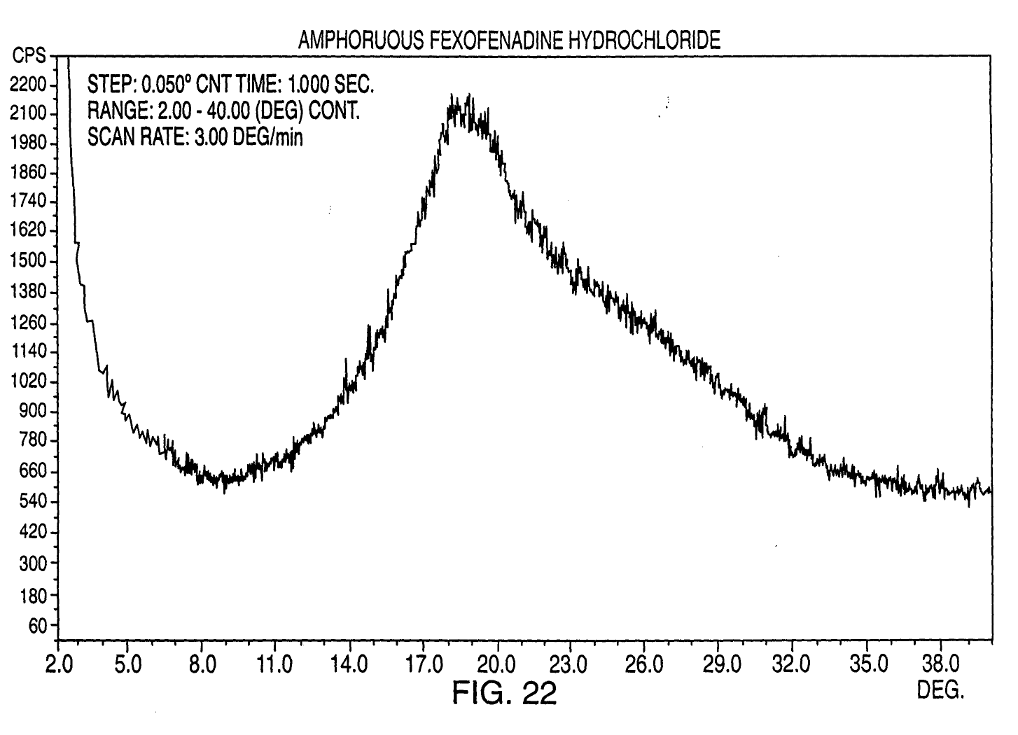

### DETAILED DESCRIPTION OF THE INVENTION

As used herein, “MTBE” refers to methyl t-butyl ether (syn. t-butyl methyl ether).

As used herein, “hydrochloride Forms VIII through XV” refers to fexofenadine hydrochloride Form VIII, hydrochloride Form IX, MTBE and cyclohexane solvates of Form IX, hydrochloride Form X, hydrochloride Form XI, hydrochloride Form XII, hydrochloride Form XIII, hydrochloride Form XIV and hydrochloride Form XV.

As used herein, “about A to B” refers to “about A to about B” unless otherwise specified.

As used herein in connection with a measured quantity, “about” refers to the normal variation in that measured quantity, as expected by the skilled artisan making the measurement and exercising a level of care commensurate with the objective of measurement and the precision of the measuring equipment. When used in relation with amount of time, “about” can have its ordinary meaning, and can be used to round the amount of time to simplify the language, for example,“about a few days” rather than “60 hours”.

As used herein, precipitation (or “precipitate”) is used in the same way as crystallization (or “crystal”), and refers to obtaining a solid material.

In one aspect, the present invention provides a process for preparing amorphous fexofenadine comprising the steps of preparing a solution of fexofenadine hydrochloride in THF, removing a portion of THF from the solution, adding a C₅ to C₁₂ saturated hydrocarbon to THF to form an upper and a lower layer, wherein the lower layer is oily, separating the upper layer from the lower layer and drying the lower layer to obtain amorphous fexofenadine.

First, a solution of fexofenadine hydrochloride in THF is prepared. To prepare the solution, fexofenadine free base can be used and converted to the hydrochloride form. The free base, for example, can be converted to the hydrochloride by being dissolved in THF and contacted with hydrochloric acid. After conversion, a portion of THF is removed from the solution, preferably by evaporation. The pressure can be reduced or the temperature increased to accelerate the evaporation process.

One skilled in the art will appreciate that removal of different volumes of THF does not necessarily change the results. Preferably, the THF is removed to a degree where its volume would be negligible compared to the volume of the saturated hydrocarbon, but not completely evaporated.

Saturated hydrocarbons generally possess lower boiling points than ethers of comparable molecular weight because of their lack of dipolarity and weaker van der walls forces. The lack of polarity makes saturated hydrocarbons a suitable solvent for extraction from a concentrated solution of fexofenadine hydrochloride in THF. Preferably, the saturated hydrocarbon is a C₅ to a C₇ saturated hydrocarbon, and most preferably, it is cyclohexane.

The addition of cyclohexane results in a two layer system, with an upper and a lower layer, wherein the lower layer is oily. The lower layer can be separated by decanting the top layer. The oily layer is then dried, resulting in amorphous fexofenadine. The oily layer can be dried under ambient or reduced pressure, or at elevated temperatures. For example, a vacuum oven known in the art can be used to dry the oily layer. The amorphous dried product may optionally be triturated with cyclohexane.

The present invention also provides a process for preparing amorphous fexofenadine hydrochloride comprising the steps of preparing a solution of fexofenadine hydrochloride in an organic solvent such as a liquid ester, ketone, alcohol or ether, and removing the solvent, such as by evaporating under ambient or reduced pressure, to obtain amorphous fexofenadine hydrochloride.

The organic solvent is preferably an ester, ketone, alcohol or ether. More preferably, the organic solvent is an alcohol, such as methanol, ethanol or isopropanol, or a ketone, such as acetone. After dissolution of fexofenadine hydrochloride in the organic solvent, the organic solvent is removed, preferably by evaporation. The solvent can be evaporated under reduced or ambient pressure. The evaporation is preferably controlled, and one skilled in the art will appreciate that the conditions of evaporation can affect the quality of the product. The final product can optionally be triturated with an organic solvent such as a saturated hydrocarbon, including inter alia cyclohexane, hexane and heptane, or ethers, including inter alia MTBE.

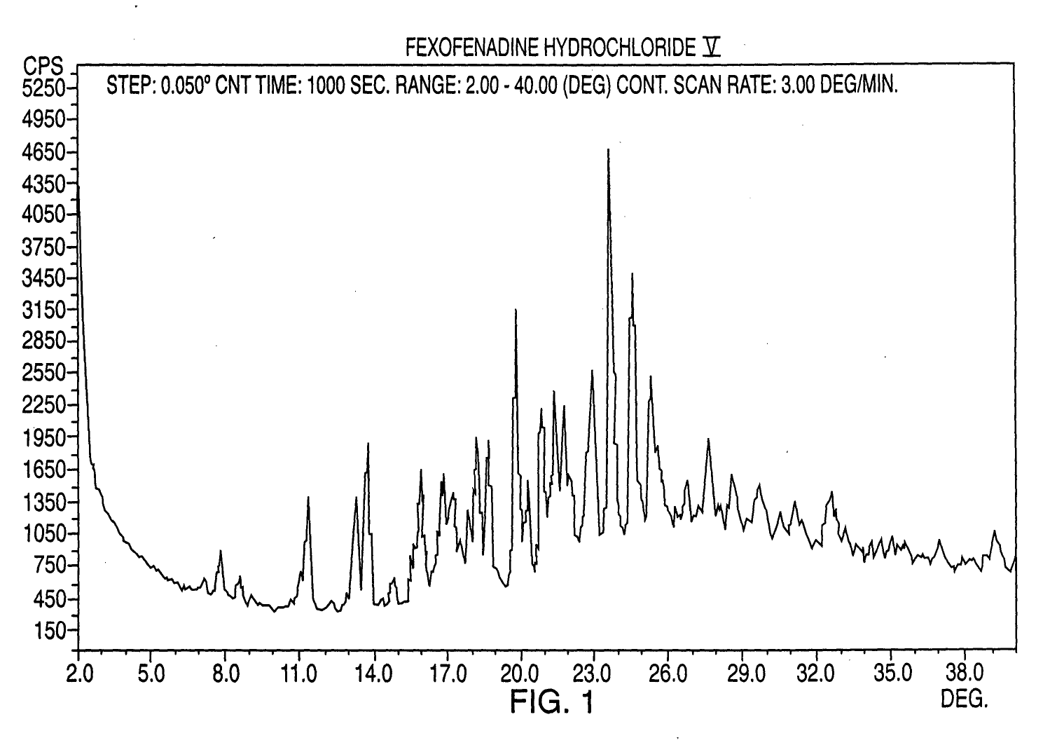

The present invention provides new crystal Form V of fexofenadine hydrochloride. Form V has peaks in the PXRD pattern (FIG. 1) at about 7.2, 7.9, 8.6, 11.0, 11.3, 13.3, 13.7, 14.8, 15.6, 15.9, 16.9, 17.2, 17.9, 18.4, 18.7, 19.9, 20.4, 20.9, 21.2, 21.5, 21.8, 22.1, 23.1, 23.8, 24.6, 25.4, 26.8, 27.7, 28.7, 29.7±0.2 degrees two theta. The most characteristic peaks are observed at about 15.9, 16.8, 17.2, 20.9, 21.5, 21.8±0.2 degrees two theta.

Karl Fischer analysis of samples of Form V shows that they can contain from about 30% to about 56% water. Form V can therefore exist in a range of hydration states. Regardless of hydration level, samples of Form V produce similar PXRD patterns, indicating that the conformation and orientation of fexofenadine undergoes little change with variation in hydration level. Therefore, while the hydration level can vary, the analytical data indicates that the characteristics that define a particular form, conformation and orientation, are not significantly changed with changes in hydration level within the 30 to 56 wt. % range.

The present invention provides a process for preparing fexofenadine hydrochloride Form V comprising the steps of preparing a solution of fexofenadine hydrochloride in a mixture of water and an alcohol selected from the group consisting of methanol, ethanol, 1-butanol and isopropanol, forming a precipitate, and separating the precipitate.

Form V can be obtained by recrystallization from a mixture of water and a lower alcohol selected from the group consisting of methanol, ethanol, 1-butanol, isopropanol and mixtures thereof. First, fexofenadine hydrochloride is dissolved in a mixture of an alcohol and water. A particularly preferred lower alcohol for obtaining Form V is ethanol. The ratio of alcohol to water is preferably from about 1:2 to about 1:10.

After crystallization, Form V can be recovered by filtration or decanting the solvent or other means. The crystals can then be washed with fresh cold recrystallization solvent or another solvent. The crystals can be dried under ambient conditions.

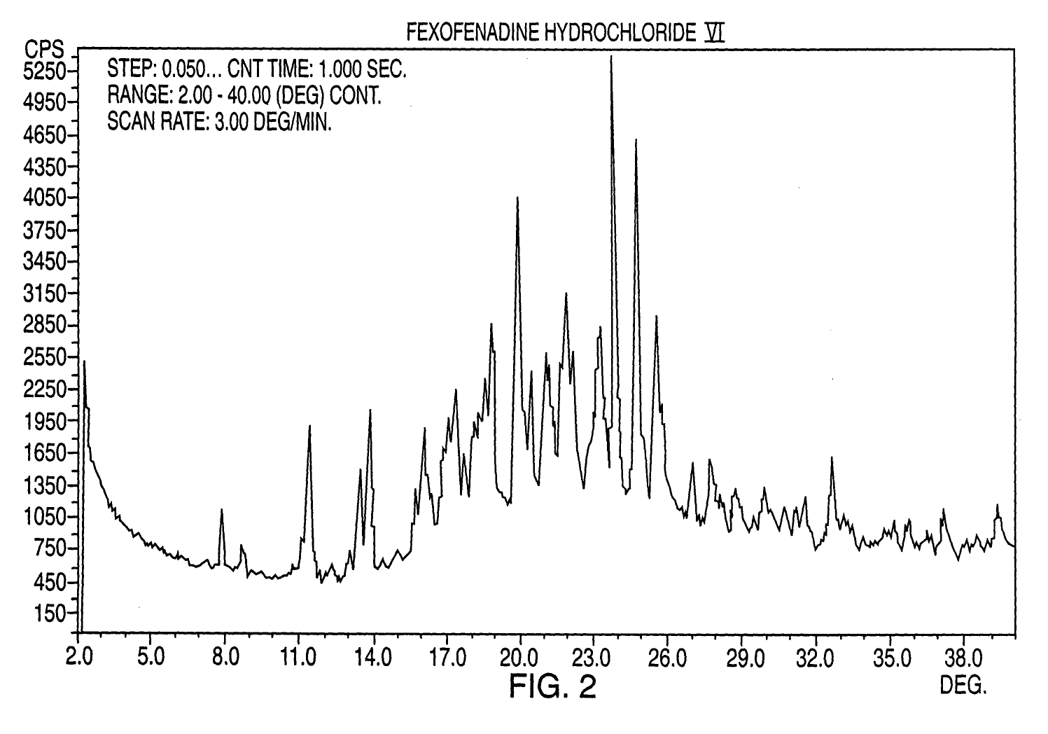

The present invention also provides new crystal Form VI of fexofenadine hydrochloride having peaks in the PXRD pattern (FIG. 2) at about 7.9, 8.7, 11.5, 13.5, 13.9, 15.7, 16.1, 17.0, 17.4, 18.1, 18.5, 18.9, 20.0, 20.5, 21.2, 21.9, 22.2, 23.3, 23.9, 24.8,25.6, 27.0, 27.9, 28.2, 28.8, 30.0, 31.2, 31.6, 32.7 degrees two theta. The most characteristic peaks are observed at about 15.7, 16.1, 17.0, 17.3, 18.6, 18.8±0.2 degrees two theta. Samples of fexofenadine hydrochloride Form VI undergo about 27% LOD by TGA analysis.

The present invention provides a process for preparing fexofenadine hydrochloride Form VI comprising the steps of preparing a solution of fexofenadine hydrochloride in a mixture of water and 1-propanol, forming a precipitate and separating the precipitate.

The process for preparing Form VI is similar to the process for preparing Form V, except that the solvent system is a mixture of water and 1-propanol, preferably in a ratio of about 2:1 to about 4:1, more preferably of about 10:3. Fexofenadine hydrochloride is dissolved in the mixture, which can be heated to obtain a clear solution. The solution is then cooled and preferably stirred. The crystals are then separated, preferably by filtration.

Fexofenadine hydrochloride Form VI also can be prepared by dissolution in THF, followed by the addition of water to cause precipitation. First, a solution of fexofenadine hydrochloride in THF is obtained. Water is then added to the solution, and a precipitate forms. After about half a day, the precipitate can be separated by techniques well known in the art, such as filtration.

The present invention also provides a process for preparing fexofenadine hydrochloride Form II by heating either fexofenadine hydrochloride Form V or Form VI. Preferably, Forms V and VI are heated to from about 40° C. to about 80° C. to induce a transition to Form II. Most preferably, they are heated at about 40° C. overnight.

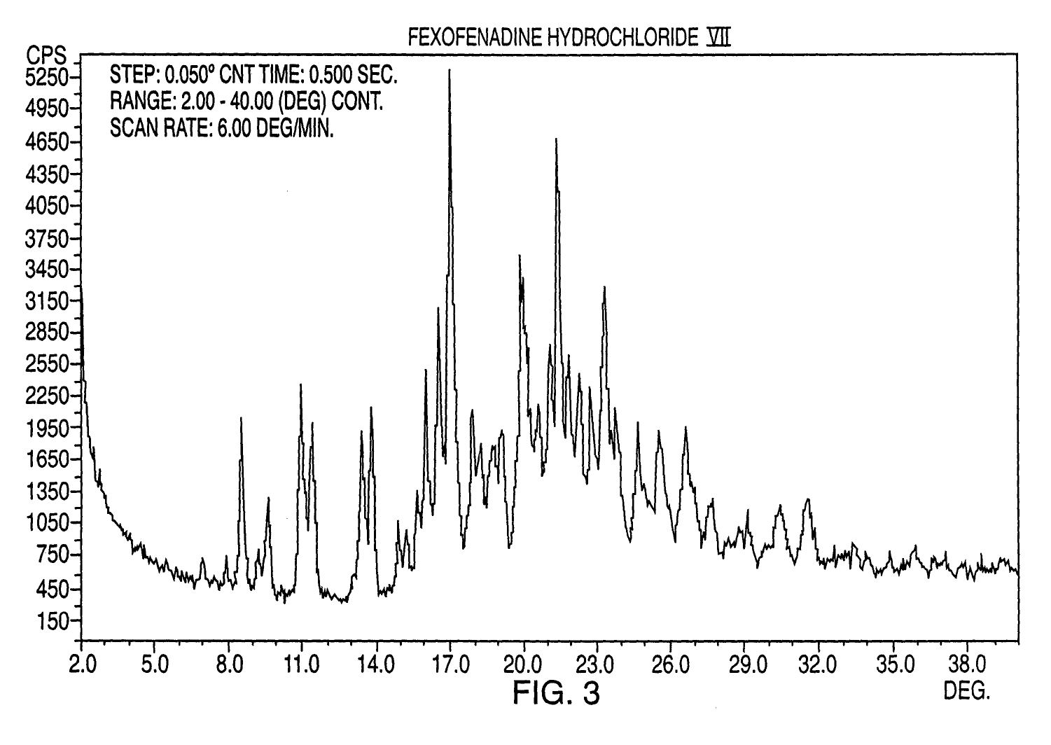

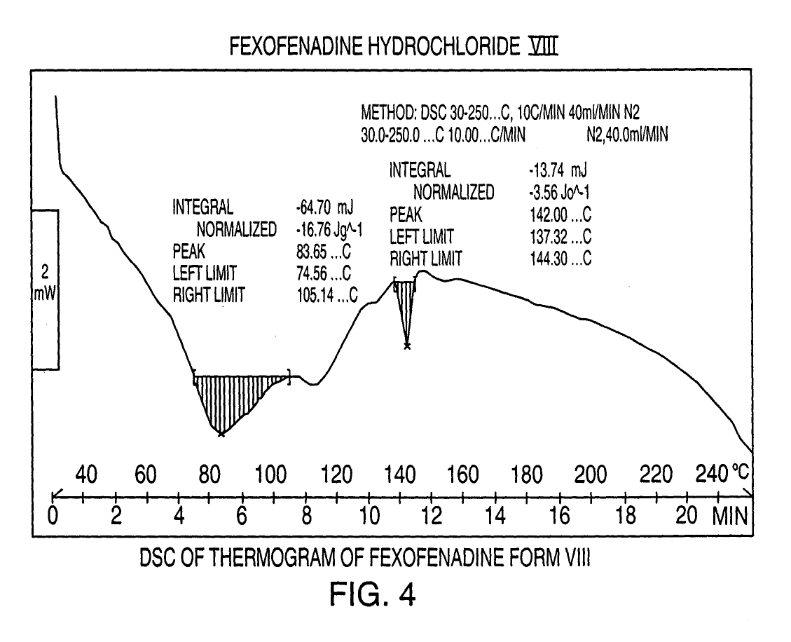

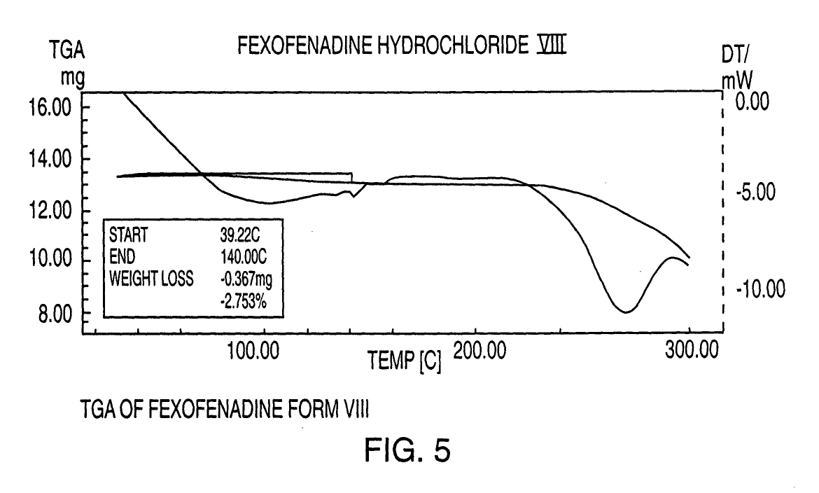

The present invention further provides new crystal Form VIII of fexofenadine hydrochloride. Fexofenadine hydrochloride Form VIII is characterized by a PXRD pattern (FIG. 3) with peaks at about 8.5, 11.0, 11.4, 13.4, 13.8, 17.1, 20.0, 21.5±0.2 degrees two theta. The DTG profile (FIG. 5) of Form VIII shows a broad multiple endothermic peak below 130° C. and gradual 2.8% weight loss between 40° C. and 140° C. At about 240° C., the weight loss accelerates, which is a result of the chemical decomposition of the sample. In addition, Form VIII is characterized by a DSC thermogram (FIG. 4) with an endothermic peak at about 84° C. and a sharp weak (3.56 J/g) endotherm at about 142° C.

The present invention provides a process for preparing fexofenadine hydrochloride Form VIII comprising the steps of preparing a solution of fexofenadine free base in a basic aqueous solvent, adding hydrochloric acid to the solution to form a precipitate and separating the precipitate.

Fexofenadine free base is first dissolved in a basic aqueous solvent such as a dilute 0.5 N solution of sodium or potassium hydroxide, preferably about 0.1 equivalents with respect to fexofenadine. The solution is then heated, preferably from about 70° C. to about 85° C., more preferably from about 75° C. to about 80° C. An excess of HCl, preferably about 1.1 to about 1.5 equivalents, with respect to fexofenadine free base is then added, said addition preferably being of a dilute solution, such as 1 N HCl, and portionwise.

After forming fexofenadine hydrochloride, the resulting mixture is cooled, preferably in an ice bath. The mixture can be stirred for about half a day. The precipitate which has formed can then be separated. Preferably, the precipitate is separated by filtration. The resulting precipitate can then be dried. Preferably, the precipitate is dried at about room temperature.

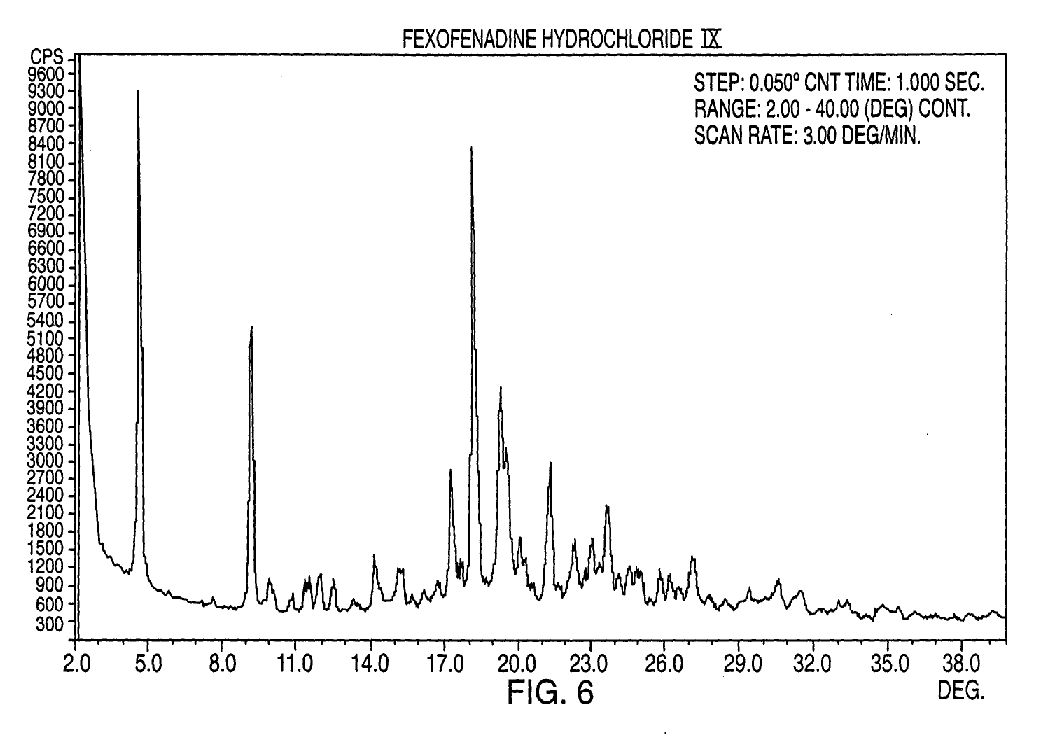

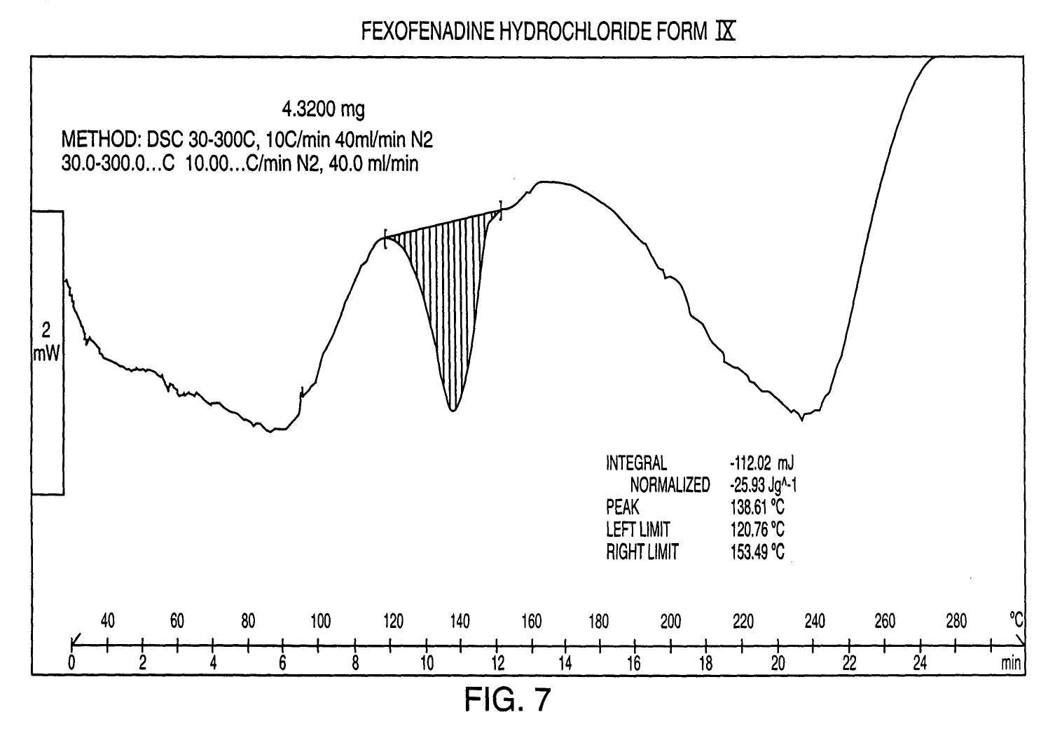

The present invention provides a new crystal form of fexofenadine hydrochloride designated Form IX. Fexofenadine hydrochloride Form IX is characterized by a PXRD pattern (FIG. 6) with peaks at about 4.7, 9.3, 17.4, 18.2, 19.4, 19.6, 21.6 and 24.0±0.2 degrees two theta. Fexofenadine hydrochloride Form IX is characterized by a DSC thermogram (FIG. 7) with an endotherm at about 139° C.

The present invention provides a process for preparing fexofenadine hydrochloride Form IX comprising the steps of preparing a solution of fexofenadine hydrochloride in acetone, adding the solution to an anti-solvent to form a precipitate and separating the precipitate.

First a solution of fexofenadine hydrochloride is prepared in acetone. To prepare the solution, fexofenadine free base is suspended in acetone, followed by contact with hydrochloric acid. To dissolve the fexofenadine hydrochloride or free base in acetone, the acetone can be heated or stirred to increase its solubility. After preparing the solution, the solution is added to the anti-solvent to form a precipitate. Preferably, the anti-solvent is an ether, wherein each alkyl radical of the ether consists of less than five carbons. Most preferably, the ether is MTBE. In another embodiment, the anti-solvent is a C₅-C₁₂ saturated hydrocarbon. Most preferably, the saturated hydrocarbon is cyclohexane.

After about half a day, the formed precipitate can be separated. Preferably, filtration is used to separate the precipitate. The precipitate also can be dried. The temperature can be increased or the pressure reduced to accelerate the drying process. Preferably, the precipitate can be dried at from about 40° C. to about 70° C. under reduced pressure.

Form IX can be crystallyzed out as a solvate of MTBE or cyclohexane. To obtain the solvates, Form IX is added to the particular solvent, and crystallized out. Crystallization out of MTBE results in a solvate of MTBE while crystallization out of cyclohexane results in a solvate of cyclohexane.

The DTG profile of Form IX-MTBE solvate is characterized by a broad endotherm at about 100° C. and an additional endotherm at about 125° C. A weight loss step is observed in the temperature range of about 115-166° C. Form IX-MTBE solvate contains about 6% MTBE. The water content of Form IX-MTBE solvate is about 3-4%, as determined by Karl Fischer.

The DTG profile of fexofenadine hydrochloride Form IX cyclohexane solvate is characterized by a broad endotherm at about 99-110° C. and an additional endotherm at about 140-150° C. The weight loss step is coincident with the second endotherm. Fexofenadine hydrochloride Form IX as a solvate of cyclohexane contains about 4.7-4.8% cyclohexane, and corresponds to a fexofenadine hydrochloride as a ⅓ solvate of cyclohexane, or 4.9%. The water content of fexofenadine hydrochloride Form IX as a solvate of cyclohexane is about 3-4%, as was determined by Karl Fischer.

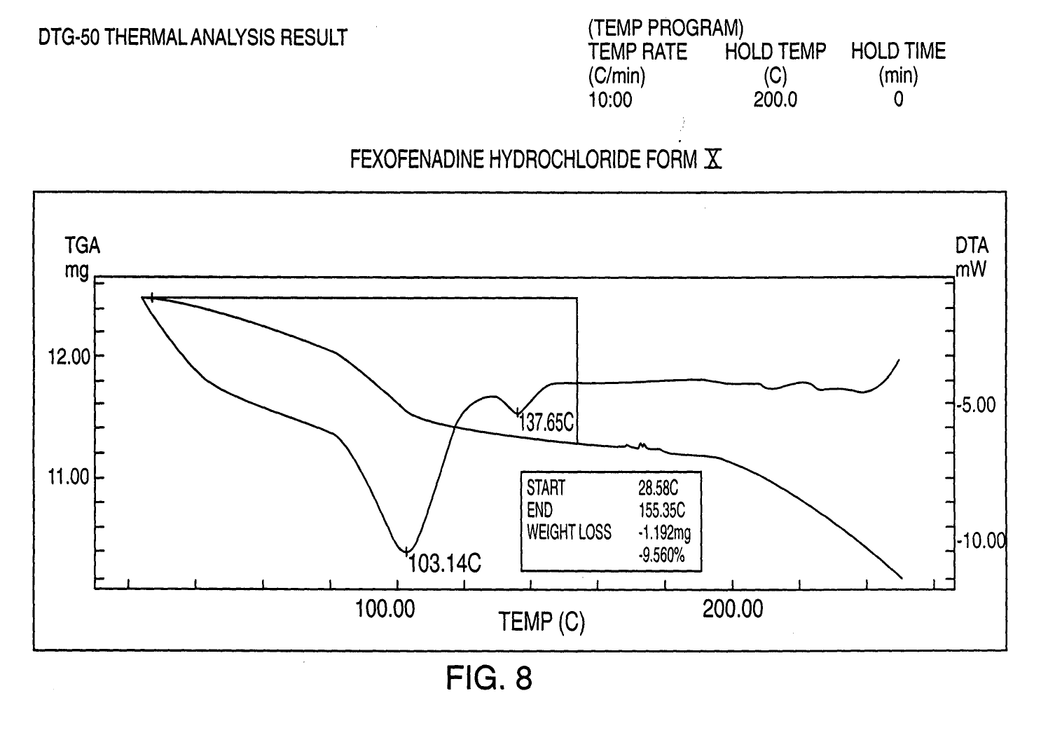

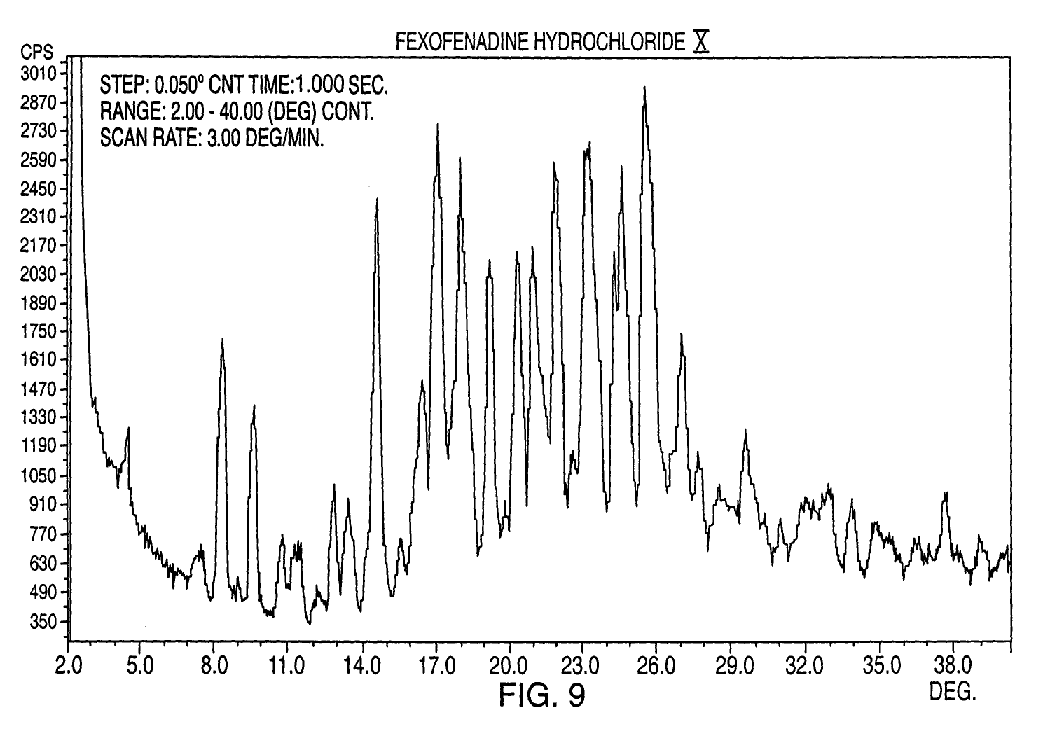

In another aspect, the present invention provides fexofenadine hydrochloride Form X (designated Form XII in Provisional Appl. No. 60/336,930, filed Nov. 8, 2001). Fexofenadine hydrochloride Form X has a PXRD pattern (FIG. 9) with characteristic peaks at about 4.2, 8.0, 9.3, 14.2, 16.0, 16.8, 17.2, 17.6, 18.8, 20.0, 20.6, 21.7, 22.9, 23.8, 24.2 and 25.4±0.2 degrees two theta. Fexofenadine hydrochloride Form X is also characterized by a DTG profile (FIG. 8) with a maximum endotherm at about 100° C. and a minor endotberm at about 138° C.

Karl Fischer analysis of wet samples of Form X show that they can contain from about 1.5 to about 8.5% water. Form X therefore can exist in a range of hydration states. Regardless of hydration level, samples of Form X produce similar PXRD patterns, indicating that the conformation and orientation of fexofenadine undergoes little change with variation in hydration level. Therefore, while the hydration level can vary, the analytical data indicates that the characteristics that define a particular form, conformation and orientation, are not significantly changed with changes in hydration level within the 1.5 to 8.5 weight % range.

The present invention also provides a process for preparing fexofenadine hydrochloride Form X comprising the steps of preparing a solution of fexofenadine hydrochloride in methanol, optionally adding dichloromethane to said solution, adding a C₅ to a C₁₂ saturated hydrocarbon to the solution to form a precipitate and separating the precipitate.

First, a solution of fexofenadine hydrochloride in methanol is prepared. To prepare the solution, fexofenadine hydrochloride is dissolved in methanol. In a preferred embodiment, dichloromethane is added to the solution.

A saturated hydrocarbon is then added to the solution to cause formation of a precipitate. Preferably, the saturated hydrocarbon is selected from the group consisting of cyclohexane and heptane. In another embodiment, the saturated hydrocarbon is added without dichloromethane to induce precipitation.

After addition of the saturated hydrocarbon, the solution can be stirred overnight. A precipitate forms which can be separated by techniques well known in the art, such as filtration. The resulting wet precipitate can be dried. The temperature can be raised or the pressure reduced to accelerate the drying process. Preferably, the precipitate is dried at about 40° C. to about 70° C. under vacuum.

In another aspect, fexofenadine hydrochloride Form X also can be prepared by a process comprising the steps of preparing a solution of fexofenadine hydrochloride in methanol, removing methanol to obtain a residue, adding a mixture of methanol and an anti-solvent to the residue to form a precipitate and separating the precipitate.

Fexofenadine hydrochloride is dissolved in methanol to prepare a solution. Methanol is then removed, preferably by evaporation, to obtain a residue. After evaporation, a mixture of methanol and an anti-solvent is added to cause formation of a precipitate. The anti-solvent can be a mono-aromatic hydrocarbon, preferably xylene or toluene. The anti-solvent also can be a C₅-C₁₂ saturated hydrocarbon, preferably heptane. The ratio of methanol to the anti-solvent is preferably from about 1:10 to about 1:30. Most preferably, it is about 1:15.

After adding the mixture to the residue, the resulting solution is preferably allowed to stand for a few hours to allow fexofenadine hydrochloride to precipitate. The precipitate is separated, preferably by filtration. The wet precipitate can be dried. To accelerate the drying process, the pressure can be reduced or the temperature increased. Preferably, the precipitate is dried at a temperature of about 40° C. to about 70° C. under reduced pressure.

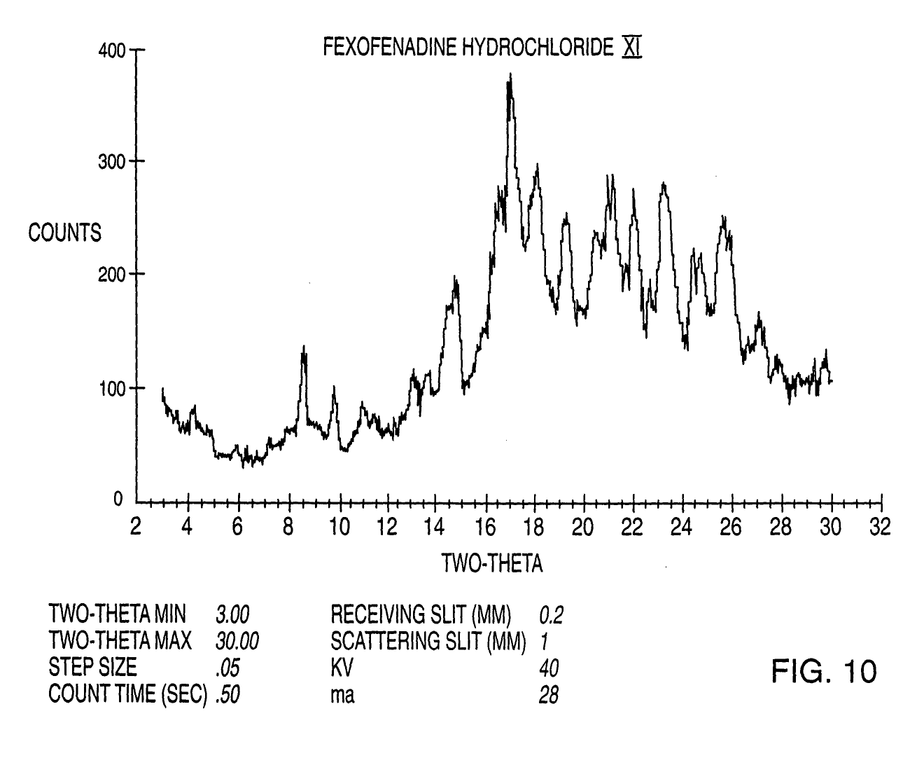

The present invention is also directed to fexofenadine hydrochloride Form XI (designated Form XIII in Provisional Application No. 60/336,930, filed Nov. 8, 2001). The fexofenadine hydrochloride Form XI has a characteristic PXRD pattern (FIG. 10) with peaks at about 8.7, 14.5, 14.9, 16.6, 17.2, 18.3, 19.5, 21.2, 22.1 and 23.3±0.2 degrees two theta.

The present invention provides a process for preparing fexofenadine hydrochloride Form XI comprising the steps of preparing a solution of fexofenadine hydrochloride in methanol, adding the solution to toluene to form a precipitate and separating the precipitate.

Fexofenadine hydrochloride is first dissolved in methanol. The solution is then added to toluene to form a precipitate. The precipitate that forms, is then separated, preferably after a few days. Preferably, filtration is used to separate the precipitate. The precipitate also can be dried. The conditions can be changed, such as a reduction in pressure or an increase in temperature, to accelerate the drying process. Preferably, the precipitate is dried at from about 40° C. to about 70° C. in a vacuum oven.

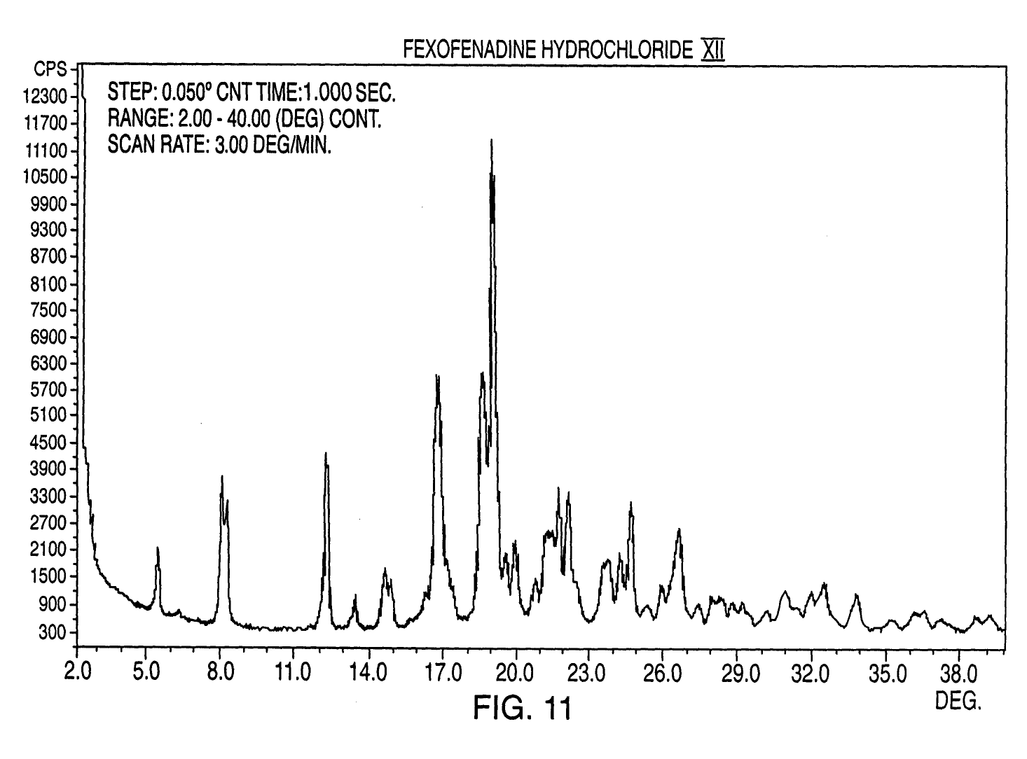

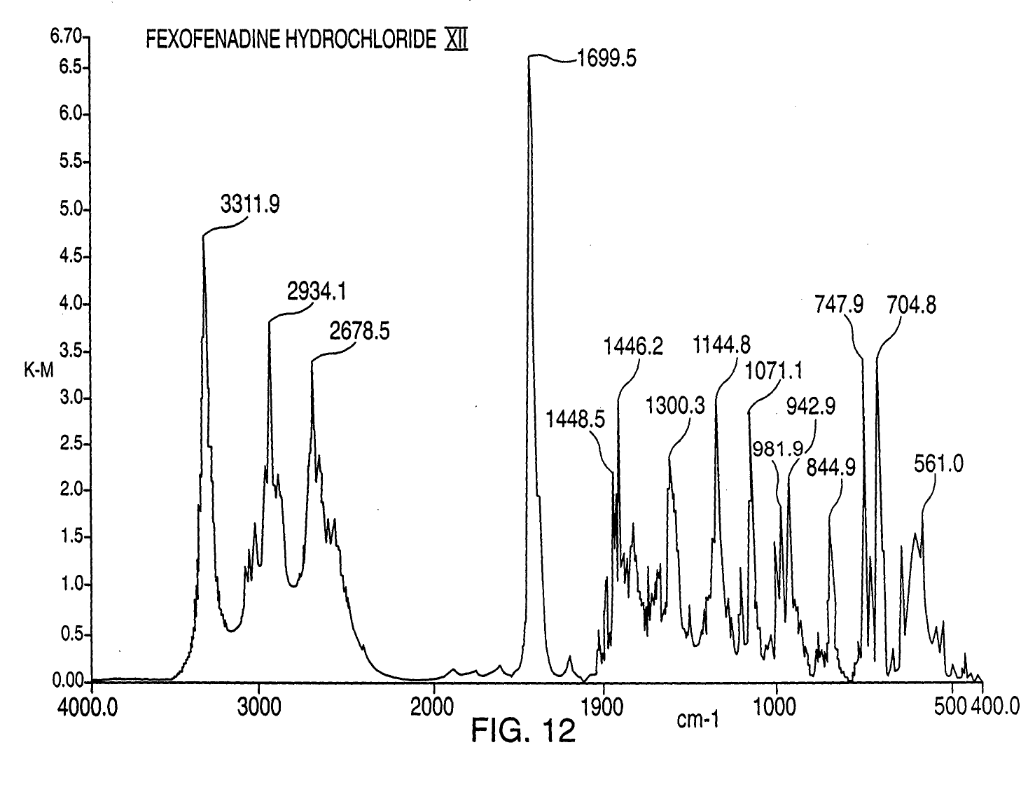

The present invention also provides fexofenadine hydrochloride Form XII. Fexofenadine hydrochloride Form XII is characterized by a PXRD pattern (FIG. 11) with peaks at about 5.2, 7.9, 8.1, 12.1, 18.5, 19.0±0.2 degrees two theta. Fexofenadine hydrochloride Form XII has a PXRD pattern with peaks at about 5.2, 7.9, 8.1, 12.1, 13.3, 14.4, 14.7, 16.6, 18.5, 19.0, 19.5, 19.8, 21.7, 22.1, 24.2, 24.6, 26.7±0.2 degrees two theta. Fexofenadine hydrochloride Form XII is also characterized by a FTIR spectrum (FIG. 12) with peaks at about 731, 845, 963, 986, 999, 1072, 1301, 1412 and 3313 cm⁻¹. The fexofenadine hydrochloride Form XII is further characterized by a FTIR spectrum with peaks at about 581, 640, 705, 748, 1165, 1337, 1367, 1448, 1468, 1700, 2679, 2934 and 3312 cm⁻¹.

The present invention provides a process for preparing fexofenadine hydrochloride Form XII comprising the steps of dissolving fexofenadine hydrochloride in ethanol to form a solution, removing ethanol to obtain a residue, adding a mixture of ethanol and toluene to the residue to form a precipitate and separating the precipitate.

Fexofenadine hydrochloride is first dissolved in ethanol. The solution can be heated or stirred to completely dissolve the fexofenadine hydrochloride. An oil bath of 50° C. can be used to heat the solution. After obtaining a homogeneous solution, the ethanol is evaporated to obtain a residue. To evaporate the ethanol, the pressure can be reduced or the temperature increased. Preferably, the temperature is from about 20° C. to about 50° C., with about 45° C. being the most preferred. The pressure can be reduced with a water aspirator, a diaphram pump or an oil pump. Preferably, a water aspirator followed by an oil pump is used. A centrifugal force can also be applied to accelerate the drying process, such as a rotovapor.

A mixture of ethanol and toluene is then added to the residue. Preferably, the mixture has a ratio of about 8:1 to about 16:1 of toluene to ethanol. The mixture containing the residue can also be stirred. A precipitate begins to form. Preferably, the mixture containing the residue is left overnight and the precipitate is separated the next day, by methods well known in the art, such as filtration. The precipitate can optionally be dried. The precipitate is preferably dried at about room temperature. Preferably, the temperature is not increased in order to avoid converting fexofenadine hydrochloride Form XII into fexofenadine hydrochloride Form XIII.

In another embodiment, before the drying step, the precipitate is suspended in heptane and heated for about 5-7 hours. The suspension is preferably heated to from about 40° C. to about 60° C., most preferably to about 50° C. After heating, the suspension is left at room temperature overnight and filtered to separate a solid. The solid is then dried, preferably for about a few hours, at a temperature of about 64° C.

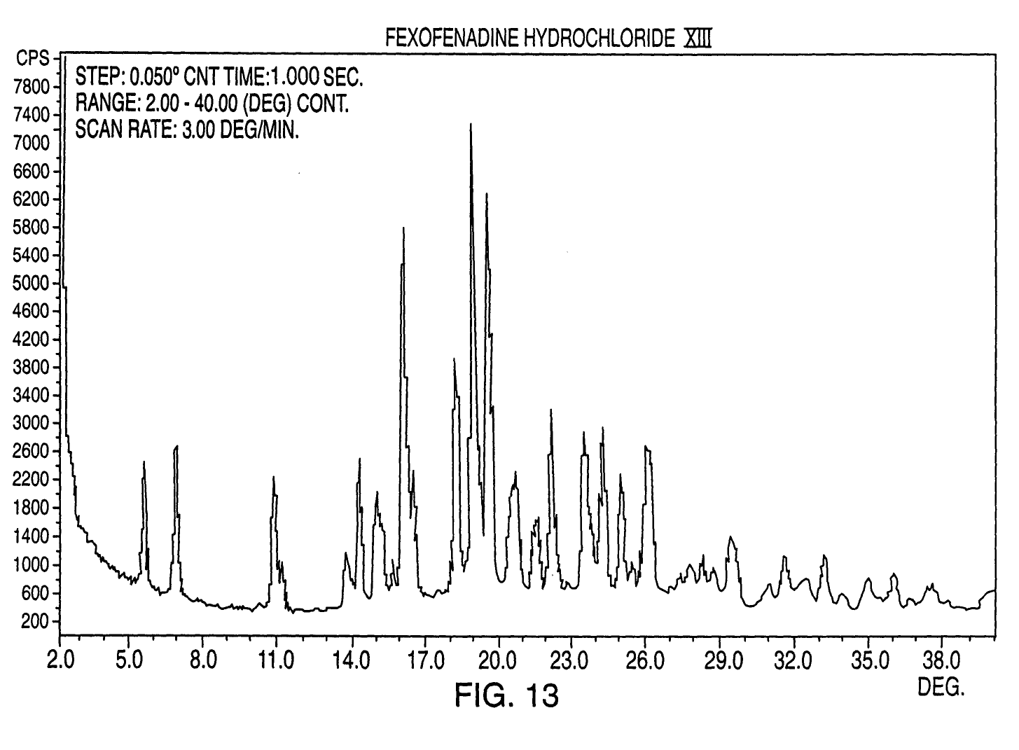

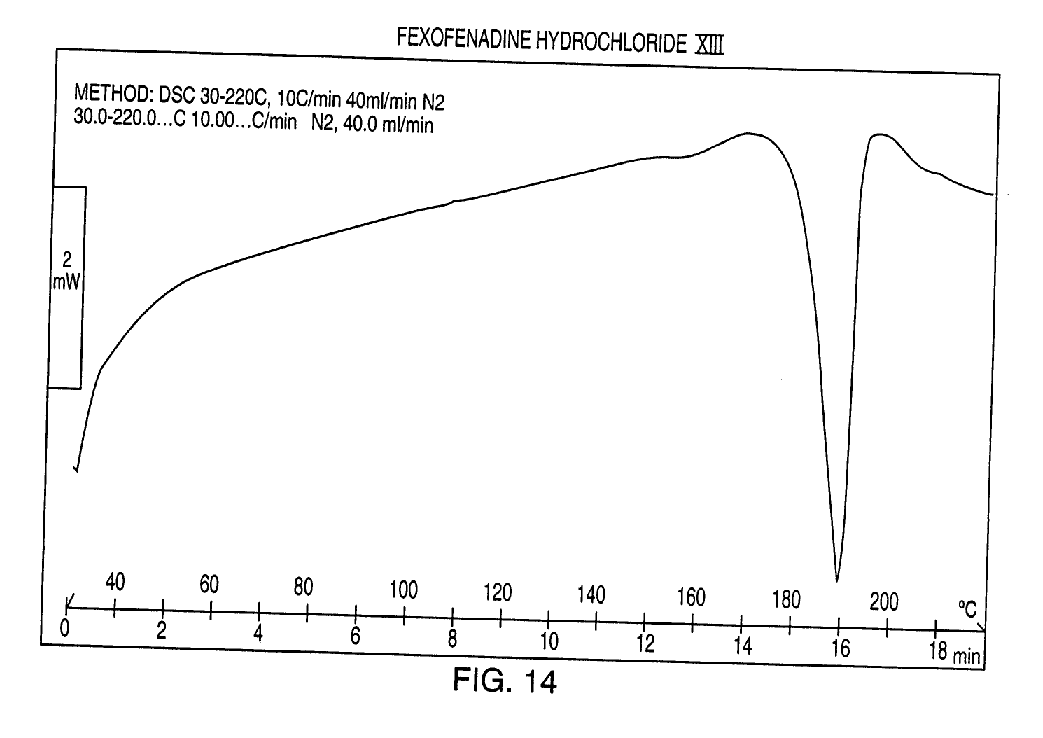

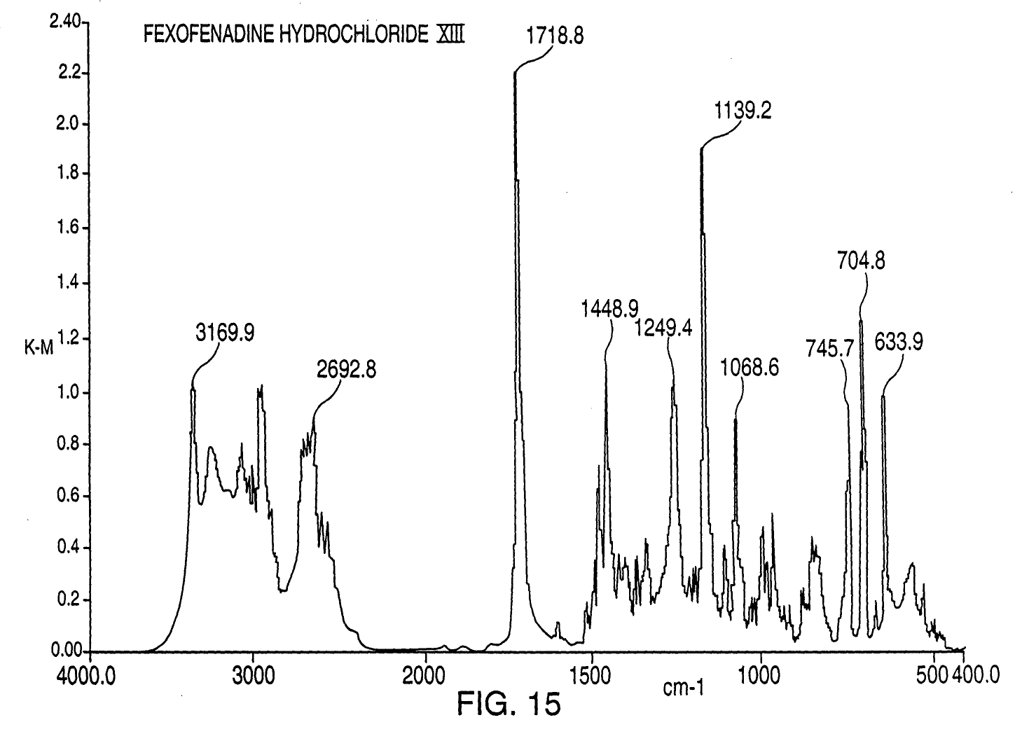

In another aspect, the present invention provides a novel crystalline form of fexofenadine hydrochloride, designated Form XIII. Fexofenadine hydrochloride Form XIII is characterized by a PXRD pattern (FIG. 13) with peaks at about 5.5, 6.8, 16.0, 16.3±0.2 degrees two theta. Fexofenadine hydrochloride Form XIII can be further characterized by a PXRD pattern with peaks at about 10.7, 11.0, 13.6, 14.2, 14.9, 18.1, 18.9, 19.5, 20.6, 21.5, 22.0, 23.4, 24.2, 24.9, 26.0±0.2 degrees two theta. Fexofenadine hydrochloride Form XIII is also characterized by a DSC thermogram (FIG. 14) with an endothermic peak at about 185-195° C. Fexofenadine hydrochloride Form XIII is also characterized by a FTIR spectrum (FIG. 15) with peaks at about 1249, 1365, 1719 and 3366 cm⁻¹. Fexofenadine hydrochloride Form XIII is further characterized by a FTIR spectrum with peaks at about 639, 705, 746, 855, 963, 995, 1069, 1159, 1449, 1474, 2653, 2681, 2949, 3067, 3261 cm⁻¹.

The present invention provides processes for preparing fexofenadine hydrochloride Form XIII from fexofenadine hydrochloride Form XII. Generally, Form XIII is prepared by heating Form XII for a sufficient amount of time. The DSC profile of Form XII is indistinguishable from the DSC profile of Form XIII.

The examples provide guidance to one skilled in the art on the amount of time and the temperature necessary to obtain Form XIII from Form XII. Heating at about 80° C. for 50 hours results in a 100% conversion to Form XIII. The preferred temperature for transformation is about 80° C. or higher, with about 95° C. to about 105° C. being the most preferred. At these high temperatures, only a few hours of heating is necessary to obtain complete transformation.

As one skilled in the art would appreciate, the process can be stopped at points during the transformation to obtain a mixture.

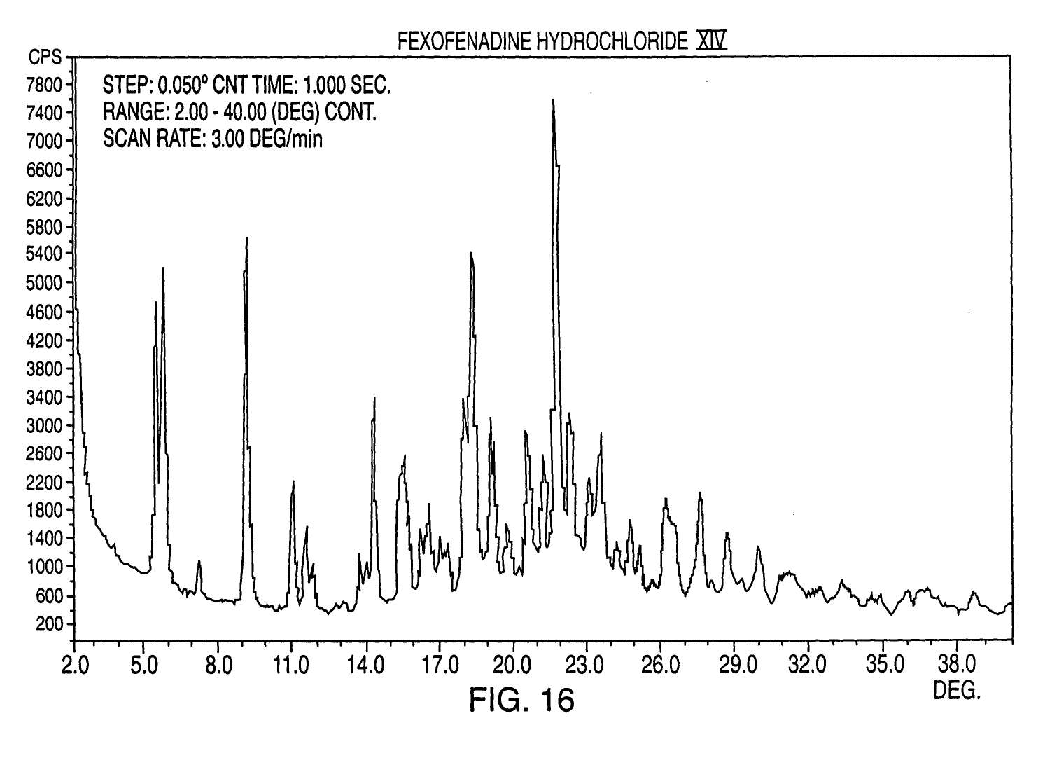

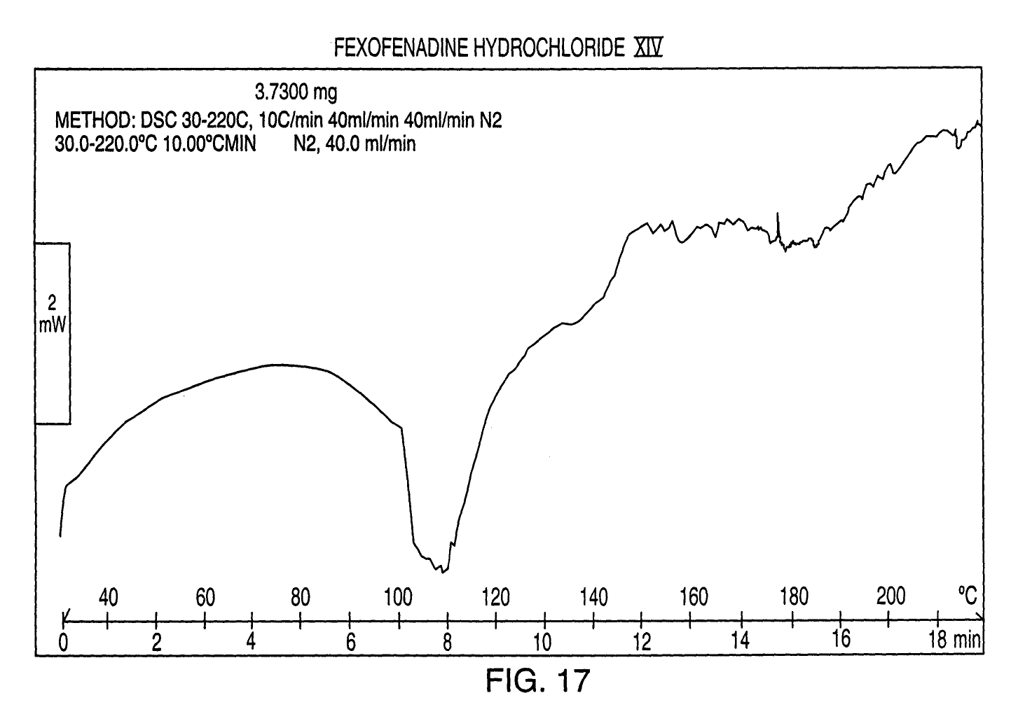

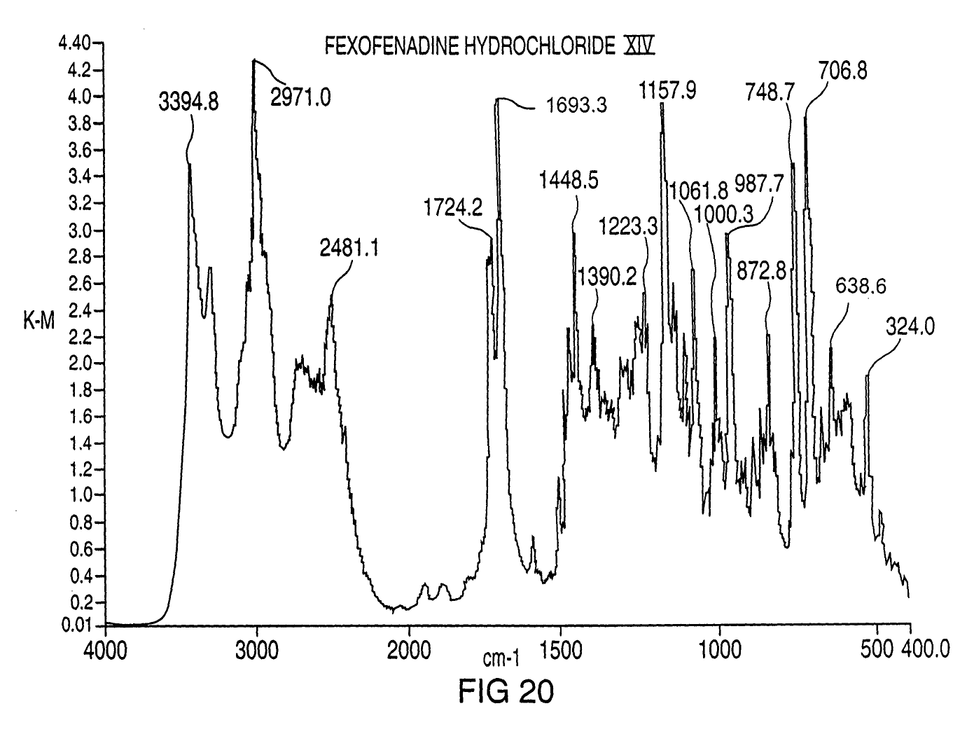

The present invention also provides fexofenadine hydrochloride as a solvate of ethyl acetate. One ethyl acetate solvate of the present invention is designated Form XIV. Fexofenadine hydrochloride ethyl acetate solvate Form XIV is characterized by a PXRD diffraction pattern (FIG. 16) with peaks at about 5.4, 5.7, 10.9, 11.4, 11.6±0.2 degrees two theta. Fexofenadine hydrochloride Form XIV is also characterized by a DSC profile (FIG. 17) with an endothermic peak at about 100° C. Fexofenadine hydrochloride ethyl acetate solvate Form XIV is also characterized by a FTIR spectrum as substantially depicted in FIG. 20.

The present invention also provides a process for preparing fexofenadine hydrochloride ethyl acetate solvate Form XIV comprising dissolving fexofenadine hydrochloride in methanol, removing methanol to obtain a residue, adding a mixture of methanol and toluene to the residue to cause formation of a precipitate, separating the precipitate, adding the precipitate to ethyl acetate to form the solvate and separating the solvate.

Fexofenadine hydrochloride is first dissolved in methanol to obtain a solution. The methanol is then evaporated to obtain a residue. Preferably, the temperature is raised and the pressure reduced to accelerate the evaporation. More preferably, the temperature is raised to from about 40° C. to about 50° C., with about 45° C. being the most preferred. The vacuum is preferably generated by using a water aspirator for about an hour, followed by a diaphram pump for a short amount of time, followed by an oil pump for a few hours. After evaporation of the methanol, a residue is obtained. A mixture of toluene and methanol is then added to the residue and the mixture is preferably stirred for a few hours. Preferably, the ratio of the mixture is about 16:1 to about 8:1 of toluene to methanol. After a few hours, a precipitate forms which is separated from the mixture of the solvents, preferably by filtration.

The precipitate is then added to ethyl acetate to obtain the solvate, though in a preferred embodiment the precipitate is dried first at about room temperature. An ice bath can be used to induce crystallization. The solvate is then separated, preferably by filtration. The solvate can also be dried. To dry, the solvate is preferably heated for a few hours from about 60° C. to about 70° C., with about 65° C. being the most preferred.

The present invention also provides for preparing Form XIV by triturating fexofenadine hydrochloride Form X in ethyl acetate. Trituration of Form X with ethyl acetate induces a transition to fexofenadine hydrochloride Form XIV.

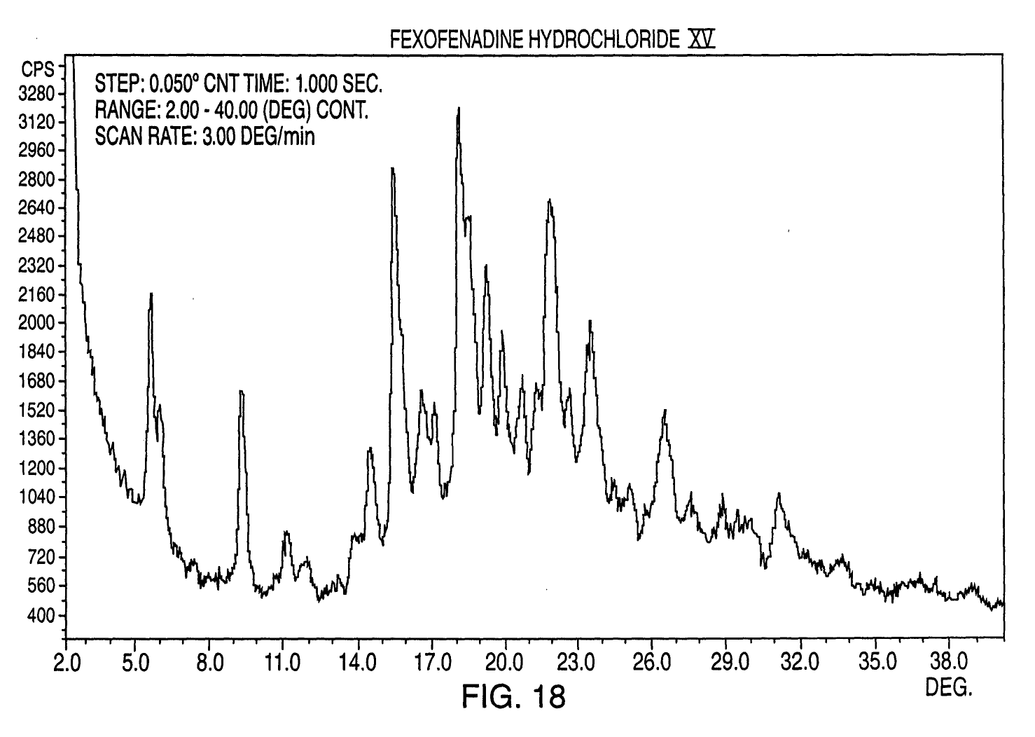

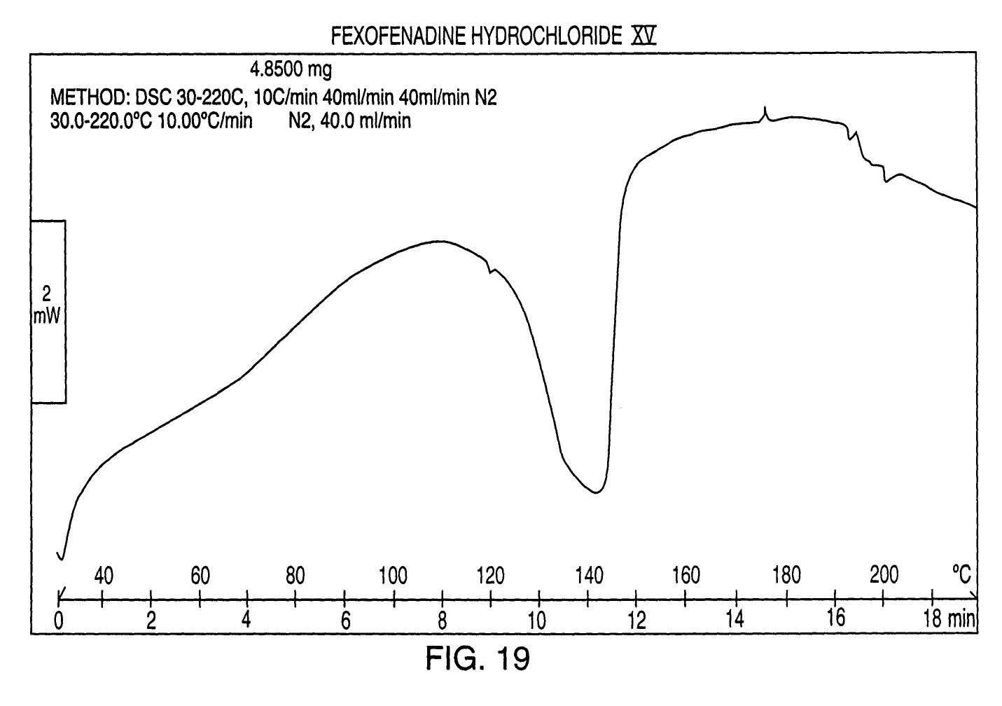

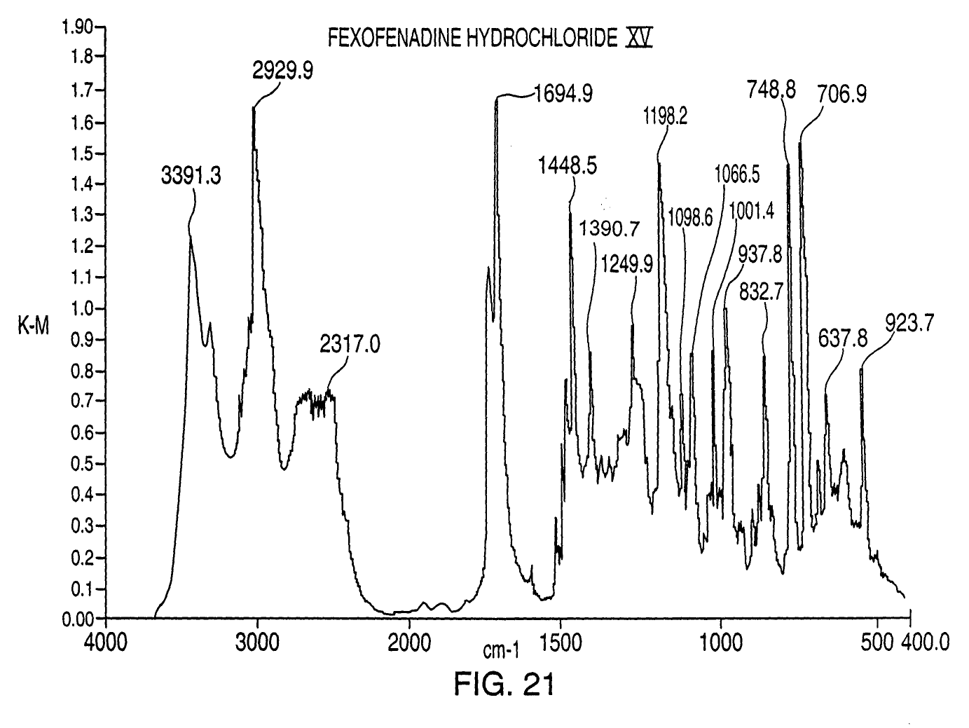

The present invention also provides a process for fexofenadine hydrochloride Form XV. Fexofenadine hydrochloride Form XV is characterized by a PXRD pattern (FIG. 18) with peaks at about 5.5, 5.8, 16.4, 16.9, 18.4±0.2 degrees two theta. Fexofenadine hydrochloride Form XV can also be characterized by a DSC thermogram (FIG. 19) with an endothermic peak at about 140° C. Fexofenadine hydrochloride Form XV is further characterized by a FTIR spectrum substantially as depicted in FIG. 21. The FTIR spectrum of Forms XIV (FIG. 20) and XV (FIG. 21) are similar, but some differences exist. The peaks at about 634.3 and about 699.5 are observed in the FTIR spectrum of Form XIV, but missing from the FTIR spectrum of Form XV. The peak at 845.3 is observed in the FTIR spectrum of Form XV as a shoulder. A splitting of the peaks is observed at 1335, 1359 and 1725 cm⁻¹ for Form XIV. Further differences also exist in the range of 2481-2522 cm⁻¹.

The present invention also provides a process for preparing fexofenadine hydrochloride Form XV comprising the steps of dissolving fexofenadine hydrochloride in ethanol, removing ethanol to obtain a residue, adding a mixture of toluene and ethanol to the residue to cause formation of a precipitate, separating the precipitate, adding the precipitate to ethyl acetate to obtain the solvate and separating the solvate.

The process for preparation of Form XV is identical to that of Form XIII in Example 32, except the last drying step is skipped, and instead of the drying step, the precipitate is stirred in ethyl acetate. The solvate crystallizes out of ethyl acetate. After forming a solvate, the solvate is separated by techniques well known in the art, such as filtration, and preferably dried overnight at a temperature of about 60° C. to about 70° C. A simple way for preparing Form XV is by triturating fexofenadine hydrochloride Form XII in ethyl acetate.

One skilled in the art would appreciate that the polymorphs of the present invention can be selectively obtained from fexofenadine hydrochloride generally through crystallization with different recrystallization solvent systems. The starting material can be anhydrous fexofenadine hydrochloride or any fexofenadine hydrochloride hydrate or lower alcohol solvate. The use of other solvates, such as the ethyl acetate solvate of the present invention, is not believed to interfere with the effectiveness of the process. The starting fexofenadine hydrochloride can also be in an amorphous or any crystalline crystal form. The process can be used as a purification method by using the desired form in an unacceptably pure state as starting material. The processes of the present invention can also be practiced as the last step in the methods discussed in U.S. Pat. Nos. 5,578,610, 5,589,487, 5,581,011, 5,663,412, 5,750,703, 5,994,549, 5,618,940, 5,631375, 5,644,061, 5,650,516, 5,652,370, 5,654,433, 5,663,353, 5,675,009, 5,375,693 and 6,147,216 to prepare a novel polymorph of the present invention.

The processes of the present invention can start with fexofenadine free base and convert the free base to the hydrochloride form. The examples and the art provide proper guidance for such conversion. Hydrochloric acid used can be aqueous or non-aqueous. The aqueous hydrochloric acid used is preferably concentrated and has a molarity of about 12 or a mass percentage of about 38%. Preferably, hydrochloric acid is used in a slight excess, more particularly from about a 1.01 to about a 1.20 molar equivalent of the free base. The free base can be regenerated by treating the salt with a suitable dilute aqueous base solution, such as dilute aqueous sodium hydroxide, potassium carbonate, ammonia or sodium bicarbonate.

Many processes of the present invention involve crystallization out of a particular solvent. One skilled in the art would appreciate that the conditions concerning crystallization can be modified without affecting the form of the polymorph obtained. For example, when mixing fexofenadine hydrochloride or free base in a solvent to form a solution, warming of the mixture can be necessary to completely dissolve the starting material. If warming does not clarify the mixture, the mixture can be diluted or filtered. To filter, the hot mixture can be passed through paper, glass fiber or other membrane material, or a clarifying agent such as celite. Depending upon the equipment used and the concentration and temperature of the solution, the filtration apparatus may need to be preheated to avoid premature crystallization.

The conditions can also be changed to induce precipitation. A preferred way of inducing precipitation is to reduce the solubility of the solvent. The solubility of the solvent can be reduced, for example, by cooling the solvent.

In one embodiment, an anti-solvent is added to a solution to decrease its solubility for a particular compound, thus resulting in precipitation. In another embodiment, an anti-solvent is added to an oily residue or a gummy material, wherein the low solubility of the anti-solvent for a particular compound results in precipitation of that compound.

Another manner to accelerate crystallization is by seeding with a crystal of the product or scratching the inner surface of the crystallization vessel with a glass rod. Other times, crystallization can occur spontaneously without any inducement. All that is necessary to be within the scope of the claims is to form a precipitate or crystal.

As an antihistamine, fexofenadine is effective at relieving symptoms caused by airborne and contact inducers of histamine release. Such substances include pollen, spores, animal dander, cockroach dander, industrial chemicals, dust and dust mites. Symptoms that can be alleviated by fexofenadine include bronchial spasms, sneezing, rhinorrhia, nasal congestion, lacrimation, redness, rash, urticaria and itch.

Fexofenadine hydrochloride Forms V, VI and VIII through XV are useful for delivering fexofenadine to the gastrointestinal tract, mucus membranes, bloodstream and inflamed tissues of a patient suffering from inflammation caused by a histamine. They can be formulated into a variety of compositions for administration to humans and animals.

Pharmaceutical compositions of the present invention contain fexofenadine hydrochloride Forms V, Form VI and Forms VIII through XV, optionally in a mixture with other forms or amorphous fexofenadine and/or active ingredients such as pseudoephedrine. They can also be optionally mixed with pseudoephedrine. In addition to the active ingredient(s), the pharmaceutical compositions of the present invention can contain one or more excipients. Excipients are added to the composition for a variety of purposes.

Diluents increase the bulk of a solid pharmaceutical composition and can make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of non-compacted solid composition and improve the accuracy of dosing. Excipients that can function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product form the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, fexofenadine hydrochloride Form V, Forms VI and Forms VIII through XV and any other solid excipients are dissolved or suspended in a liquid carrier such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at levels safe for ingestion to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches and losenges as well as liquid syrups, suspensions and elixirs.

A dosage form of the present invention is a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

Capsules, tablets and lozenges and other unit dosage forms preferably contain a dosage level of about 30 to about 180 mg of fexofenadine hydrochloride. Other dosages may also be administered depending on the need.

The following describes the instrumentation used by the present invention to characterize the new polymorphs. PXRD patterns were obtained by methods known in the art using a Scintag X-ray powder diffractometer, a variable goniometer, an X-Ray tube with Cu target anode (Cu radiation λ=1.5418 Å) and a solid state detector. A round standard aluminum sample holder with a round zero background quartz plate was used. Scans were performed over a range of 2 to 40 degrees two-theta, continuously, with a scan rate of 3 degrees/min.

Some of the samples were preformed on Philips XRD, a Goniometer Model 1050/70, a copper tube and a curved graphite monochromate. Same scanning parameters were used.

To obtain the FTIR results, we utilized a Perkin-Elmer Spectrum One FTIR spectrometer, using the diffuse reflectance technique. The spectrum was recorded from 4000-400 cm⁻¹. Sixteen scans were taken at a resolution of 4.0 cm⁻¹.

The DSC thermogram was obtained using a DSC Mettler 821 Star. The temperature range of scans was 30-350° C. at a rate of 10° C./min. The weight of the sample was 2-5 mg. The sample was purged with nitrogen gas at a flow rate of 40 mL/min. Standard 40 μl aluminum crucibles having lids with three small holes were used.

The DTG Profile for TGA analysis was obtained by a Shimadzv DTG-50, a heating rate of 10° C./minute, nitrogen flow of 20 mL/minute, a sample weight of 7-15 mg, and allumina pan.

### EXAMPLES

### Example 1

Preparation of Amorphous Fexofenadine Hydrochloride

Fexofenadine free base (8.5 grams) was dissolved in THF (850 mL). Gaseous HCl was bubbled into the solution. Afterwards, the THF and the excess of HCL were evaporated until a small volume (70 mL) was achieved. Cyclohexane (230 mL) is then added, forming an upper layer and an oily layer. The upper layer was decanted and the oily layer was evaporated until dryness. The resulting foam was triturated with cyclohexane and filtered. The wet product was dried at 50° C. overnight.

### Example 2

Preparation of Amorphous Fexofenadine Hydrochloride

Fexofenadine hydrochloride (2 grams) was dissolved in methanol (5 mL) and then evaporated until dryness. The dry material was triturated with cyclohexane (15 mL) and filtered. The wet product was dried at 50° C. overnight.

### Example 3

Preparation of Amorphous Fexofenadine Hydrochloride

Fexofenadine hydrochloride was dissolved in methanol (25 mL) to form a solution. The methanol was then evaporated under vacuum, resulting in amorphous fexofenadine.

### Example 4

Preparation of Amorphous Fexofenadine Hydrochloride

Fexofenadine hydrochloride was dissolved in ethanol (25 mL) to form a solution. The methanol was then evaporated under vacuum, resulting in amorphous fexofenadine.

### Example 5

Preparation of Amorphous Fexofenadine Hydrochloride

Fexofenadine hydrochloride was dissolved in isopropanol (25 mL) to form a solution. The methanol was then evaporated under vacuum, resulting in amorphous fexofenadine.

### Example 6

Preparation of Amorphous Fexofenadine Hydrochloride

Fexofenadine hydrochloride was dissolved in acetone (25 mL) to form a solution. The methanol was then evaporated under vacuum, resulting in amorphous fexofenadine.

### Example 7

Preparation of Fexofenadine Hydrochloride Form V

Fexofenadine hydrochloride (10 g) was suspended in a 5:1 water:ethanol mixture (100 mL) and heated until it dissolved. The solution was left to cool and the crystals were collected by filtration.

### Example 8

Preparation of Fexofenadine Hydrochloride Form V

Fexofenadine hydrochloride (10 g) was added to a 3:10 methanol:water mixture (130 mL) and heated until it dissolved. The solution was left to cool under stirring and the crystals were collected by filtration.

### Example 9

Preparation of Fexofenadine Hydrochloride Form V

Fexofenadine hydrochloride (10 g) was added to a 3:10 1-butanol:water mixture (130 mL) and heated to until it dissolved. The solution was left to cool under stirring and the crystals were collected by filtration.

### Example 10

Preparation of Fexofenadine Hydrochloride Form V

Fexofenadine hydrochloride (10 g) was added to a 3:10 isopropanol:water mixture (130 mL) and heated to until it dissolved. The solution was left to cool under stirring and the crystals were collected by filtration.

### Example 11

Preparation of Fexofenadine Hydrochloride Form VI

Fexofenadine hydrochloride (10 g) was added to a 3:10 1-propanol:water mixture (130 ml) and heated to until it dissolved. The solution was left to cool under stirring and the crystals were collected by filtration.

### Example 12

Preparation of Fexofenadine Hydrochloride Form VI

Fexofenadine hydrochloride (10 g) was suspended in THF. One equivalent of concentrated hydrochloric acid was added. A clear solution was formed. Water was then added and a precipitate formed. After 16 hours, the suspension was filtered.

### Example 13

Preparation of Fexofenadine Hydrochloride Form VIII

Fexofenadine free base (5.1 g) was dissolved in 20 mL of 0.5N NaOH aqueous solution and was heated to 75-80° C. using a hot water bath while stirring. 1N HCl aqueous solution (15 mL) was added in portions to the hot solution. The resulting mixture was stirred overnight without heating and cooled afterwards in an ice-water bath, then filtered and dried at room temperature.

### Example 14

Preparation of Fexofenadine Hydrochloride Form IX

Fexofenadine HCl (5 grams) was dissolved in boiling acetone (5 mL) while stirring. Cyclohexane (10 mL) was then added and a viscous precipitate appeared. Acetone (2.5 mL) was then added. The sample was stirred overnight at room temperature. The precipitating crystals were filtered and dried at 65° C. under vacuum.

### Example 15

Preparation of Fexofenadine Hydrochloride Form IX

Fexofenadine free base (5 grams) was suspended in boiling acetone (10 mL). Concentrated aqueous HCl solution (1.2 mL) was added. The resulting solution was added dropwise to cyclohexane (50 mL) while stirring. The sample was stirred overnight. The crystals were filtered and dried at 65° C. under vacuum.

### Example 16

Preparation of Fexofenadine Hydrochloride Form IX

Fexofenadine free base (5 grams) was suspended in boiling acetone (5 mL) while stirring. Concentrated aqueous HCL solution (1.2 mL) was added. The resulting solution was added dropwise to tertbuylmethylether (50 mL) while stirring. The solution is stirred for a couple of hours and then the crystals are filtered and dried at 65° C. under vacuum.

### Example 17

Preparation of Fexofenadine Hydrochloride Form X

Fexofenadine hydrochloride (5 grams) was dissolved in a minimum amount of methanol (10 mL). The methanol was then fully evaporated to obtain a solid material. The solid was taken up in a mixture of toluene (28 mL) and methanol (2 mL) and allowed to stand over a few hours after which time a precipitate was formed. After few hours, the precipitate was filtered, resulting in fexofenadine hydrochloride Form X as a wet sample. A portion of the wet fexofenadine hydrochloride Form X was also dried under vacuum at 65° C. Subsequent PXRD analysis of both the wet and dried portions showed that they were a new form of fexofenadine hydrochloride, labeled Form X.

### Example 18

Preparation of Fexofenadine Hydrochloride Form X

Fexofenadine hydrochloride (5 grams) was dissolved in a minimum amount of methanol (10 mL). The methanol was then fully evaporated to obtain a solid material. The solid was taken up in a mixture of xylene (28 mL) and methanol (2 mL) and allowed to stand over a few hours after which time a precipitate was formed. After few hours, the precipitate was filtered, resulting in fexofenadine hydrochloride Form X as a wet sample. A portion of the wet fexofenadine hydrochloride Form X was also dried under vacuum at 65° C. Subsequent PXRD analysis of both the wet and dried portions showed that they were a new form of fexofenadine hydrochloride, labeled Form X.

Xylene used in this Example was a mixture of various forms of xylene and was purchased from Fautarom.

### Example 19

Preparation of Fexofenadine Hydrochloride Form X

Fexofenadine hydrochloride (5 grams) was dissolved in a minimum amount of methanol (10 mL). The methanol was then fully evaporated to obtain a solid material. The solid was taken up in a mixture of heptane (28 mL) and methanol (2 mL) and allowed to stand over a few hours after which time a precipitate was formed. After few hours, the precipitate was filtered, resulting in fexofenadine hydrochloride Form X as a wet sample. A portion of the wet fexofenadine hydrochloride Form X was also dried under vacuum at 65° C. Subsequent PXRD analysis of both the wet and dried portions showed that they were a new form of fexofenadine hydrochloride, labeled Form X.

### Example 20

Preparation of Fexofenadine Hydrochloride Form X

Fexofenadine HCl (7.5 grams) was dissolved in methanol (15 mL) to form a solution. Dichloromethane (25 mL) was added to the solution, followed by cyclohexane (60 mL). The solvents were partially evaporated. The solution was then stirred by a magnetic stirrer overnight, which led to formation of precipitate. The precipitate was filtered, resulting in fexofenadine hydrochloride Form X as a wet sample. A portion of the wet fexofenadine hydrochloride Form X was also dried under vacuum at 65° C. Subsequent PXRD analysis of both the wet and dried portions showed that they were a new form of fexofenadine hydrochloride, labeled Form X.

### Example 21

Preparation of Fexofenadine Hydrochloride Form X

Fexofenadine hydrochloride (7.5 grams) was dissolved in methanol (15 mL). Dichloromethane (30 mL) was added to the solution followed by heptane (30 mL). The solvents were partially evaporated, and then the solution was stirred by a magnetic stirrer overnight, which formed a precipitate. The precipitate was filtered, resulting in fexofenadine hydrochloride Form X as a wet sample. A portion of the wet fexofenadine hydrochloride Form X was also dried under vacuum at 65° C. Subsequent PXRD analysis of both the wet and dried portions showed that they were a new form of fexofenadine hydrochloride, labeled Form X.

### Example 22

Preparation of Fexofenadine Hydrochloride Form X

Fexofenadine hydrochloride (5 grams) was dissolved in methanol (5 mL) to form a solution. The solution was then added to cyclohexane (50 mL) with vigorous stirring. The solution was left for two days, resulting in precipitation of crystals. The crystals were then filtered. Subsequent PXRD analysis confirmed that the product was a new form of fexofenadine hydrochloride, labeled Form X.

### Example 23

Preparation of Fexofenadine Hydrochloride Form XI

Fexofenadine hydrochloride (5 grams) was dissolved in methanol (5 mL). The solution was then precipitated by adding the solution to toluene (50 mL) with vigorous stirring. After two days, the precipitate was filtered and dried in a vacuum oven at 65° C. Subsequent PXRD analysis confirmed that the product was a new form of fexofenadine hydrochloride, labeled Form XI.

### Example 24

Preparation of Fexofenadine Hydrochloride Form XII

Fexofenadine hydrochloride (8 grams) was dissolved with heating in absolute ethanol (50 mL). The solution was evaporated in the rotovapor to dryness with a bath temperature of 50° C. A mixture of toluene (44 mL) and ethanol (4 mL) was added and it was stirred overnight. It was cooled in an ice bath, filtered, and washed with toluene. Subsequent PXRD analysis confirmed that the product was a new form of fexofenadine hydrochloride, labeled fexofenadine hydrochloride Form XII.

### Example 25

Preparation of Fexofenadine Hydrochloride Form XII

Fexofenadine hydrochloride (15 grams) was dissolved in 105 mL ethanol in a 250 mL round bottom flask with magnetic stirring and slight heating. The ethanol was evaporated off with a water aspirator for 1½ hours and a bath temperature of 44° C. and then afterwards with a diaphram pump for ½ hour and then with an oil pump for ½ hour. The resulting material was scrapped out of the flask and divided into 5 and 10 gram portions. The 5 gram portion was suspended in a mixture of toluene (28 mL) and ethanol (2.25 mL) in an oil bath at 50° C. After 2 hours it crystallized. It was left to stir overnight. The next day it was cooled to room temperature, filtered and dried at room temperature. Subsequent PXRD analysis confirmed that the product was a new form of fexofenadine hydrochloride, labeled fexofenadine hydrochloride Form XII.

### Example 26

Preparation of Fexofenadine Hydrochloride Form XII

The 10 gram portion from the previous example was suspended in a mixture of 4.5 mL ethanol and 56 mL toluene and stirred overnight a room temperature. The material which crystallized out was filtered, suspended in heptane in an oil bath at 50° C. for 5-7 hours and then left overnight without heating. The solid was filtered and dried for 2 hours at 64° C. Subsequent PXRD analysis confirmed that the product was a new form of fexofenadine hydrochloride, labeled fexofenadine hydrochloride Form XII.

### Example 27

Preparation of Fexofenadine Hydrochloride Form XII

Fexofenadine hydrochloride (10 grams) was magnetically stirred in 70 mL of ethanol in a 100 mL round bottom flask in an oil bath at 50° C. until it dissolved. It was connected to a water aspirator for 1 hour and then to an oil pump overnight. The next day, 55 mL toluene and 6 mL ethanol were added with stirring, and was left to stir overnight and then filtered. Subsequent PXRD analysis confirmed that the product was a new form of fexofenadine hydrochloride, labeled fexofenadine hydrochloride Form XII.

### Example 28

Preparation of a Mixture of Fexofenadine Hydrochloride Form XII and XIII

Fexofenadine hydrochloride Form XII (prepared as in Example 24) was dried at 65° C. for 2 hours, which resulted in a mixture of Form XII and Form XIII. Subsequent PXRD analysis confirmed that the existence of the mixture.

### Example 29

Preparation of Fexofenadine Hydrochloride Form XIII

Fexofenadine hydrochloride Form XII (prepared as in Example 24) was dried for 2 hours at 95-105° C., which resulted in fexofenadine hydrochloride Form XIII. Subsequent PXRD analysis confirmed that the product was a new form of fexofenadine hydrochloride, labeled fexofenadine hydrochloride Form XIII.

### Example 30

Preparation of Fexofenadine Hydrochloride Form XIII

Fexofenadine hydrochloride Form XII (prepared as in Example 25) was dried at 64° C., which resulted in a mixture of Form XII and Form XIII. Subsequent PXRD analysis confirmed that the existence of the mixture.

### Example 31

Preparation of Fexofenadine Hydrochloride Form XIII

Fexofenadine hydrochloride Form XII (prepared as in Example 28) was dried for 2 hours at 53° C., which resulted in a mixture of form XII and form XIII. Subsequent PXRD analysis confirmed the existence of the mixture. A mixture of fexofenadine hydrochloride Form XII and form XIII (prepared as in previous procedure) was dried at 1-2 mm Hg for 2 hours at 63° C., which resulted in fexofenadine hydrochloride Form XIII. Subsequent PXRD analysis confirmed that the product was a new form of fexofenadine hydrochloride, labeled fexofenadine hydrochloride Form XIII.

### Example 32

Preparation of Fexofenadine Hydrochloride Form XIII

Fexofenadine hydrochloride (20 grams) was dissolved in ethanol (140 mL) in a 250 mL round bottom flask with slight heating. The ethanol was distilled off first with a water aspirator and then with an oil pump with an oil bath temperature of 45° C. Toluene (110 mL) and ethanol (12 mL) was added with stirring. After 2 hours a precipitate became visible. Filtered after 7 hours. Part of the precipitate (3 grams) was dried at 63° C. for 24 hours under vacuum from an oil pump. Subsequent PXRD analysis confirmed that the product was a new form of fexofenadine hydrochloride, labeled fexofenadine hydrochloride Form XIII.

### Example 33

Preparation of Fexofenadine Hydrochloride ethyl acetate solvate Form XIV

Fexofenadine hydrochloride (20 grams) was dissolved in methanol (44 mL) in a 250 mL flask with stirring. The methanol was evaporated under vacuum in an oil bath at 44° C. under vacuum. First with a water aspirator for 45 minutes and then with a diaphram pump for 15 minutes and then with an oil pump for 3 hours. A mixture of toluene (112 mL) and methanol (9 mL) was then added and it was stirred for several hours. It was filtered and allowed to dry at room temperature over the weekend, after which 3 grams of the dried material was suspended in ethyl acetate, in which it dissolved. The solution was stirred for 2 hours in an ice bath. A precipitate formed, which was filtered and dried for 2-3 hours at 64° C. Subsequent PXRD analysis confirmed that the product was a new form of fexofenadine hydrochloride, labeled fexofenadine hydrochloride ethyl acetate solvate Form XIV.

### Example 34

Preparation of Fexofenadine Hydrochloride ethyl acetate solvate Form XV

Fexofenadine hydrochloride (20 grams) was dissolved in ethanol (140 mL) in a 250 mL round bottom flask with slight heating. The ethanol was distilled off first with a water aspirator and then with an oil pump with an oil bath temperature of 45° C. Then toluene (110 mL) and ethanol (12 mL) was added with stirring. After 2 hours, a precipitate became visible and was filtered after 7 hours. The filtered material (3 grams) was stirred with ethyl acetate (15 mL), filtered and dried overnight with an oil vacuum pump at 63° C. Subsequent PXRD analysis confirmed that the product was a new form of fexofenadine hydrochloride, labeled fexofenadine hydrochloride ethyl acetate solvate Form XV.

### Example 35

Preparation of Fexofenadine Hydrochloride Form II

Fexofenadine hydrochloride Form V is heated to 40° C. overnight to obtain fexofenadine hydrochloride Form II.

### Example 36

Preparation of Fexofenadine Hydrochloride Form II

Fexofenadine hydrochloride Form VI is heated to 40° C. overnight to obtain fexofenadine hydrochloride Form II.

### Example 37

Preparation of Fexofenadine Hydrochloride

Fexofenadine free base (6.5 grams, 12.1 mmole) was put in a 100 ml Erlenmeyer flask with a magnetic stirrer. The flask was put in a hot water bath and 2.2 ml of 36% HCl (25.3 mmoles) in THF (10 mL) was added. Everything dissolved. Water was added portionwise. After 10 mls of water, the mixture became cloudy, after an additional 15 mls, an oily emulsion was obtained. This was allowed to stir overnight at room temperature. The next day, the mixture was granular. An additional 25 mls of water were added and it was stirred first at room temperature and then cooled in an ice bath. It was filtered and washed with a small amount of water and put into a bottle while still wet.

Having thus described the invention with reference to particular preferred embodiments and illustrated it with examples, those in the art will appreciate modifications to the invention as described and illustrated that do not depart from the spirit and scope of the invention as disclosed in the specification.

What is claimed is:

1. A process for preparing amorphous fexofenadine hydrochloride comprising the steps of:
a) preparing a solution of fexofenadine hydrochloride in THF;
b) removing a portion of THF from the solution;
c) adding a C₅ to a C₁₂ saturated hydrocarbon to the remaining THF to form an upper and a lower layer, wherein the lower layer is oily;
d) separating the upper layer from the lower layer; and
e. drying the lower layer to obtain amorphous fexofenadine hydrochloride.

2. The process of claim 1, wherein the saturated hydrocarbon is cyclohexane.

3. The process of claim 1, wherein the volume of THF after removal is negligible compared to the volume of the less polar organic solvent.

4. The process of claim 1, wherein THF is removed by evaporation.

5. The process of claim 1, wherein the lower layer is dried by evaporation.

6. A process for preparing amorphous fexofenadine hydrochloride comprising:
a) preparing a solution of fexofenadine hydrochloride in an organic solvent; and
b) removing the solvent to obtain amorphous fexofenadine hydrochloride.

7. The process of claim 6, wherein the organic solvent is selected from the group consisting of an ester, ether, alcohol and ketone.

8. The process of claim 7, wherein the alcohol is selected from the group consisting of methanol, ethanol and isopropanol.

9. The process of claim 6, wherein the ketone is acetone.

10. The process of claim 6, wherein the solvent is removed by evaporation.

11. Fexofenadine hydrochloride Form V.

12. Fexofenadine hydrochloride of claim 11 chracterized by a water content of from about 30 to about 56%.

13. Fexofenadine hydrochloride having a PXRD pattern with peaks at about 15.9, 16.8, 17.2, 20.9, 21.5, 21.8±0.2 degrees two theta.

14. The fexofenadine hydrochloride of claim 13, having a PXRD pattern with peaks at about 7.2, 7.9, 8.6, 11.0, 13.7, 14.8, 15.6, 16.9, 17.2, 17.9, 18.4, 18.7, 19.9, 20.4, 20.9, 21.2, 21.5, 21.8, 22.1, 23.1, 23.8, 24.6, 25.4±0.2 degrees two theta.

15. The fexofenadine hydrochloride Form V of claim 14 that produces a PXRD pattern substantially as depicted in FIG. 1.

16. A process for preparing fexofenadine hydrochloride Form V comprising the steps of:
a) preparing a solution of fexofenadine hydrochloride in a mixture of water and an alcohol selected from the group consisting of methanol, isopropanol, ethanol and 1-butanol;
b) precipitating fexofenadine hydrochloride from the solution; and
c) separating the precipitate.

17. The process of claim 16, wherein the mixture has a ratio of about 2:1 of water to alcohol.

18. Fexofenadine hydrochloride Form VI.

19. Fexofenadine hydrochloride having a PXRD pattern with peaks at about 15.7, 16.1, 17.0, 17.3, 18.6, 18.8±0.2 degrees two theta.

20. The fexofenadine hydrochloride of claim 19 that produces a PXRD pattern with peaks at about 7.2, 7.9, 8.6, 11.0, 11.3, 13.3, 13.7, 14.8, 15.6, 15.9, 16.1, 16.9, 17.0, 17.2, 17.3, 17.9, 18.4, 18.6, 18.7, 19.9, 20.4, 20.9, 21.2, 21.5, 21.8, 22.1, 23.1, 23.8, 24.6, 25.4, 26.8, 27.7, 28.7, 29.7±0.2 degrees two theta.

21. The fexofenadine hydrochloride of claim 20 that produces a PXRD pattern substantially as depicted in FIG. 2.

22. A process for preparing fexofenadine hydrochloride Form VI comprising the steps of:
a) preparing a solution of fexofenadine hydrochloride in a mixture of water and 1-propanol;
b) precipitating fexofenadine hydrochloride from the solution; and
c) separating the precipitate.

23. The process of claim 22, wherein the mixture is from about a 2:1 to about a 4:1 mixture of water and 1-propanol.

24. A process for preparing fexofenadine hydrochloride Form VI comprising the steps of:
a) preparing a solution of fexofenadine hydrochloride in THF;
b) adding water to the solution to form a precipitate; and
c) separating the precipitate.

25. A process for preparing fexofenadine hydrochloride Form II comprising heating fexofenadine hydrochloride selected from the group consisting of Form V and Form VI to a temperature of from about 40° C. to about 80° C.

26. The process of claim 25, wherein the temperature is about 40° C.

27. Fexofenadine hydrochloride Form VIII.

28. Fexofenadine hydrochloride having a PXRD pattern with peaks at 8.5, 11.0, 11.4, 13.4, 13.8, 17.1, 20.0, 21.5±0.2 degrees two theta.

29. The fexofenadine hydrochloride of claim 28 having a PXRD pattern substantially as depicted in FIG. 3.

30. Fexofenadine hydrochloride having a DSC thermogram with endothermic peaks at about 84° C. and about 142° C.

31. Fexofenadine hydrochloride having a DTG profile as depicted in FIG. 5.

32. A process for preparing fexofenadine hydrochloride Form VIII comprising the steps of:
a) preparing a solution of fexofenadine free base in a basic aqueous solvent;
b) adding hydrochloric acid to the solution to form a precipitate; and
c) separating the precipitate.

33. The process of claim 32, further comprising a step of drying the precipitate.

34. Fexofendine hydrochloride Form IX.

35. Fexofenadine hydrochloride characterized by a PXRD pattern with peaks at about 4.7, 9.3, 17.4, 18.2, 19.4, 19.6, 21.6 and 24.0±0.2 degrees two theta.

36. The fexofenadine hydrochloride of claim 35 having a PXRD pattern substantially as depicted in FIG. 6.

37. Fexofenadine hydrochloride characterized by a differential scanning calorimetry endothermic peak at about 139° C.

38. A process for preparing fexofenadine hydrochloride Form IX comprising the steps of:
a) preparing a solution of fexofenadine hydrochloride in acetone;
b) adding the solution to an anti-solvent to form a precipitate; and
c) separating the precipitate.

39. The process of claim 38, further comprising drying the precipitate.

40. The process of claim 38, wherein the anti-solvent is an ether.

41. The process of claim 40, wherein the ether is MTBE.

42. The process of claim 38, wherein the anti-solvent is a C₅-C₁₂ saturated hydrocarbon.

43. The process of claim 42, wherein the saturated hydrocarbon is cyclohexane.

44. Fexofenadine hydrochloride MTBE solvate.

45. Fexofenadine hydrochloride MTBE solvate of claim 44 characterized by a DTG profile with endotherms at about 100° C. and about 125° C.

46. A process for preparing fexofenadine hydrochloride MTBE solvate comprising the steps of:
a) adding fexofenadine hydrochloride Form IX to MTBE to form the solvate; and
b) separating the solvate.

47. Fexofenadine hydrochloride cyclohexane solvate.

48. The solvate of claim 47 characterized by a DTG profile with endotherms at about 99° C. to about 110° C. and about 140° C. to about 150° C.

49. A process for preparing fexofenadine hydrochloride cyclohexane solvate comprising the steps of:
a) adding fexofenadine hydrochloride Form IX to cyclohexane to form the solvate; and
b) separating the solvate.

50. Fexofenadine hydrochloride Form X.

51. The fexofenadine hydrochloride of claim 50 having a water content of about 7.5 to 8.5%.

52. Fexofenadine hydrochloride having a PXRD pattern with peaks at about 4.2, 8.0, 9.3, 14.2, 16.0, 16.8, 17.6, 18.8, 20.0, 20.6, 21.7, 22.9, 23.8, 24.2 and 25.4±0.2 degrees two theta.

53. The fexofenadine hydrochloride of claim 52 having a PXRD pattern substantially as depicted in FIG. 9.

54. Fexofenadine hydrochloride characterized by a DTG profile with a maximum endotherm at about 100° C. and a minor endotherm at about 138° C.

55. A process for preparing fexofenadine hydrochloride Form X comprising the steps of:
a) preparing a solution of fexofenadine hydrochloride in methanol, and optionally adding dichloromethane to said solution;
b) adding a C₅ to a C ₂ saturated hydrocarbon to the solution to form a precipitate; and
c) separating the precipitate.

56. The process of claim 55, further comprising drying the precipitate.

57. The process of claim 55, wherein the saturated hydrocarbon is selected from the group consisting of cyclohexane and heptane.

58. The process of claim 55, further comprising a step of drying the precipitate.

59. A process for preparing fexofenadine hydrochloride Form X comprising the steps of:
a) preparing a solution of fexofenadine hydrochloride in methanol;
b) removing methanol to obtain a residue;
c) adding a mixture of methanol and an anti-solvent to the residue to form a precipitate; and
d) separating the precipitate.

60. The process of claim 59, wherein the anti-solvent is a monoaromatic hydrocarbon.

61. The process of claim 60, wherein the monoaromatic hydrocarbon is selected from the group consisting of toluene and xylene.

62. The process of claim 59, wherein the anti-solvent is a C₅ to a C₁₂ saturated hydrocarbon.

63. The process of claim 62, wherein the anti-solvent is heptane.

64. The process of claim 59, further comprising drying the precipitate.

65. Fexofenadine hydrochloride Form XI.

66. Fexofenadine hydrochloride having a PXRD pattern with peaks at about 8.7, 14.5, 14.9, 16.6, 17.2, 18.3, 19.5, 21.2, 22.1 and 23.3±0.2 degrees two theta.

67. The fexofenadine hydrochloride of claim 66 having a PXRD pattern substantially as depicted in FIG. 10.

68. A process for preparing fexofenadine hydrochloride Form XI comprising the steps of:
a) preparing a solution of fexofenadine hydrochloride in methanol;
b) adding the solution to toluene to form a precipitate; and
c) separating the precipitate.

69. The process of claim 68, further comprising drying the precipitate.

70. Fexofenadine hydrochloride Form XII.

71. Fexofenadine hydrochloride that produces a PXRD pattern with peaks at about 5.2, 7.9, 8.1, 12.1, 18.5, 19.0±0.2 degrees two theta.

72. The fexofenadine hydrochloride of claim 71 wherein the PXRD pattern has peaks at about 5.2, 7.9, 8.1, 12.1, 13.3, 14.4, 14.7, 16.6, 18.5, 19.0, 19.5, 19.8, 21.7, 22.1, 24.2, 24.6, 26.7±0.2 degrees two theta.

73. The fexofenadine hydrochloride of claim 72 characterized by a PXRD pattern substantially as depicted in FIG. 11.

74. Fexofenadine hydrochloride characterized by a FTIR spectrum with peaks at about 731, 845, 963, 986, 999, 1072, 1301, 1412 and 3313 cm⁻¹.

75. The fexofenadine hydrochloride of claim 74 further characterized by a FTIR spectrum with peaks at about 581, 640, 705, 748, 1165, 1337, 1367, 1448, 1468, 1700, 2679, 2934 and 3312 cm⁻¹.

76. The fexofenadine hydrochloride of claim 75 characterized by a FTIR spectrum substantially as depicted in FIG. 12.

77. A process for preparing fexofenadine hydrochloride Form XII comprising the steps of:
a) preparing a solution of fexofenadine hydrochloride in ethanol;
b) removing ethanol to obtain a residue;
c) adding a mixture of ethanol and toluene to the residue to form a precipitate; and
d) separating the precipitate.

78. The process of claim 77, wherein ethanol is removed by evaporation.

79. The process of claim 77, wherein the mixture has a ratio of about 8:1 to about 16:1 of toluene to ethanol.

80. The process of claim 77, further comprising drying the precipitate.

81. Fexofenadine hydrochloride Form XIII.

82. Fexofenadine hydrochloride characterized by a PXRD pattern with peaks at about 5.5, 6.8, 16.0, 16.3±0.2 degrees two theta.

83. The fexofenadine hydrochloride of claim 82 wherein the PXRD pattern has peaks at about 5.5, 6.8, 10.7, 11.0, 13.6, 14.2, 14.9, 16.0, 16.3, 18.1, 18.9, 19.5, 20.6, 21.5, 22.0, 23.4, 24.2, 24.9, 26.0±0.2 degrees two theta.

84. The fexofenadine hydrochloride of claim 83 characterized by a PXRD pattern substantially as depicted in FIG. 13.

85. Fexofenadine hydrochloride characterized by a DSC thermogram with an endothermic peak at about 185-195° C.

86. Fexofenadine hydrochloride characterized by a FTIR spectrum with peaks at about 1249, 1365, 1719 and 3366cm⁻¹.

87. The fexofenadine hydrochloride of claim 86 having a FTIR spectrum with peaks at about 639, 705, 746, 855, 963, 995, 1069, 1159, 1249, 1365, 1449, 1474, 1719, 2653, 2681, 2949, 3067, 3261 and 3366cm⁻¹.

88. The fexofenadine hydrochloride of claim 87 characterized by a FTIR spectrum as substantially depicted in FIG. 15.

89. A process for preparing fexofenadine hydrochloride Form XIII by heating fexofenadine hydrochloride Form XII for a sufficient amount of time to obtain Form XIII.

90. The process of claim 89, wherein fexofenadine hydrochloride Form XII is heated to a temperature of at least about 80° C.

91. The process of claim 89, wherein the process is stopped before complete transformation to fexofenadine hydrochloride Form XIII to obtain a mixture of Form XII and Form XIII.

92. A fexofenadine hydrochloride ethyl acetate solvate.

93. Fexofenadine hydrochloride ethyl acetate solvate Form XIV.

94. Fexofenadine hydrochloride ethyl acetate solvate characterized by a PXRD diffraction pattern with peaks at about 5.4, 5.7, 10.9, 11.4, 11.6±0.2 degrees two theta.

95. The fexofenadine hydrochloride ethyl acetate solvate of claim 94 characterized by a PXRD pattern substantially as depicted in FIG. 16.

96. Fexofenadine hydrochloride ethyl acetate solvate characterized by a DSC thermogram with an endothermic peak at about 100° C.

97. Fexofenadine hydrochloride ethyl acetate solvate characterized by a FTIR spectrum with peaks at about 634.3, 699.5, 1335, 1359 and 1725 cm⁻¹, wherein the peaks at 1335, 1359 and 1725 are split.

98. The fexofenadine hydrochloride ethyl acetate solvate of claim 97 having a FTIR spectrum substantially as depicted in FIG. 20.

99. A process for preparing fexofenadine hydrochloride ethyl acetate solvate Form XIV comprising:
a) dissolving fexofenadine hydrochloride in methanol;
b) removing methanol to obtain a residue;
c) adding a mixture of methanol and toluene to the residue to form a precipitate;
d) separating the precipitate;
e) adding the precipitate to ethyl acetate to form the solvate; and
f) separating the solvate.

100. The process of claim 99, wherein methanol is removed by evaporation.

101. The process of claim 99, further comprising a step of drying the solvate.

102. The process of claim 99, wherein the ratio of the mixture is about 8:1 to about 14:1 of toluene to methanol.

103. A process for preparing fexofenadine hydrochloride ethyl acetate solvate Form XIV comprising triturating fexofenadine hydrochloride From X in ethyl acetate.

104. Fexofenadine hydrochloride ethyl acetate solvate Form XV.

105. Fexofenadine hydrochloride characterized by a PXRD pattern with peaks at about 5.5, 5.8, 16.4, 16.9, 18.4±0.2 degrees two theta.

106. The fexofenadine hydrochloride of claim 105 having a PXRD pattern substantially as depicted in FIG. 18.

107. Fexofenadine hydrochloride characterized by a DSC thermogram with an endotherm at about 140° C.

108. Fexofenadine hydrochloride ethyl acetate solvate characterized by a FTIR spectrum as substantially depicted in FIG. 21.

109. A process for preparing fexofenadine hydrochloride ethyl acetate solvate Form XV comprising the steps of:
a) dissolving fexofenadine hydrochloride in ethanol;
b) removing ethanol to obtain a residue;
c) adding a mixture of toluene and ethanol to the residue to form a precipitate;
d) separating the precipitate;
e) adding the precipitate to ethyl acetate to form the solvate; and
f) separating the solvate.

110. The process of claim 109, wherein ethanol is removed by evaporation.

111. The process of claim 109, further comprising drying the solvate.

112. The process of claim 109, wherein the mixture has a ratio of 8:1 to 14:1 of toluene to ethanol.

113. A process for preparing fexofenadine hydrochloride Form XV comprising triturating fexofenadine hydrochloride Form XII in ethyl acetate.

114. A pharmaceutical composition comprising:
a) fexofenadine hydrochloride selected from the group consisting of Form V, Form VI, Form VIII, Form IX, Form IX-MTBE solvate, Form IX-cyclohexane solvate, Form X, Form XI, Form XII, Form XIII, Form XIV and Form XV; and
b) a pharmaceutically acceptable excipient.

115. A pharmaceutical dosage form comprising the pharmaceutical composition of claim 114.

116. The pharmaceutical dosage form of claim 115, wherein the dosage form is a capsule or tablet.

117. A unit dosage of the pharmaceutical dosage form of claim 115 containing about 30 to about 180 mg of fexofenadine hydrochloride.

118. A method of inhibiting binding between an H₁ receptor and histamine in a mammal comprising administering the pharmaceutical composition of claim 114 to the mammal.

119. The method of claim 118, wherein the mammal has symptoms selected from the group consisting of contraction of the bronchi, vasodilation, excessive mucus as result of inflammation and itching.

120. A method of alleviating symptoms of allergic rhinitis in a patient susceptible to allergic rhinitis or experiencing symptoms of allergic rhinitis comprising administering to the patient the pharmaceutical composition of claim 114.
